(19) United States
(12) Patent Application Publication
(10) Pub. No.: US 2026/0265699 A1
(43) Pub. Date: Sep. 10, 2026

(54) STANDARDISING PLURIPOTENT STEM CELLS

(71) Applicant: United Kingdom Research and Innovation, Wiltshire (GB)

(72) Inventors: Madeline A. LANCASTER, Cambridge (GB); Magdalena SUTCLIFFE, Sawston (GB)

(21) Appl. No.: 19/491,430

(22) PCT Filed: Jun. 13, 2024
(86) PCT No.: PCT/EP2024/066492

§ 371 Date: Dec. 9, 2025

(30) Foreign Application Priority Data
Jun. 13, 2023 (GB) 2308828.9

(51) Int. Cl. C12N 5/074; A61K 35/545

(52) U.S. Cl. CPC C12N 5/0696; A61K 35/545; C12N 2501/72

(57) ABSTRACT

The invention relates to a method of standardising pluripotent stem cells (PSCs) comprising culturing at least one PSC in the presence of an inhibitor of a H3K9 HMTase, to standardised cells produced as a result of carrying out this method, and to the use of these cells in protocols to generate derivatives of all three germ layers.

### FIELD OF THE INVENTION

The invention relates to a method of standardising pluripotent stem cells (PSCs). In particular, the method comprises culturing at least one PSC in the presence of an inhibitor of a methyltransferase that catalyses the mono-, di- and tri-methylated states of histone H3 at lysine residue 9 (H3K9 HMTase), a H3K9 HMTase inhibitor. Other agents that affect epigenetic landscape of chromatin may also be present. The invention extends to standardised cells produced as a result of carrying out this method, and to the use of these cells in protocols to generate derivatives of all three germ layers, including cerebral organoids, and in disease modelling.

### BACKGROUND OF THE INVENTION

In multicellular organisms, stem cells are undifferentiated or partially differentiated cells that can differentiate into various types of cells and proliferate indefinitely to produce more of the same stem cell. Stem cells are a powerful tool for modelling human development and disease, for use in drug discovery, and for use in the development of cell therapies.

One of the earliest stem cell types are PSCs. Pluripotency refers to a stem cell that has the potential to differentiate into any of the three germ layers: endoderm (gut, lungs, yolk sac), mesoderm (muscle, skeleton, blood, vascular, urogenital, dermis), or ectoderm (nervous, sensory, epidermis), but not into extra-embryonic tissue like the placenta. Embryonic stem cells (ESC), derived from the blastocyst stage of early mammalian embryos are a type of PSC.

Induced pluripotent stem cells (iPSCs) are a type of PSC artificially derived from a non-pluripotent cell, typically an adult somatic cell, by inducing forced expression of various genes and transcription factors. Due to their similarity to ESC, the medical and research communities are extremely interested in iPSCs.

A cerebral organoid or brain organoid is an artificially grown miniature organ resembling the brain. Their production involves an advanced method of neural differentiation from pluripotent stem cells. They can model many aspects of early brain development, defects associated with neurodevelopmental and neurological disorders, and aid in the discovery of new therapeutics for these diseases.

Similarly, a renal organoid or kidney organoid, and a digestive tract or intestinal organoid, are artificially grown miniature organs resembling the kidney or intestines, respectively. Their production also involves differentiation from pluripotent stem cells and they can function as early developmental models aiding in the study of defects and disorders and the discovery of new treatments.

Due, in part, to their ability to both self-renew and differentiate into different cell types, PSCs (including naturally occurring PSC, iPSCs and ESCs) and the products of their differentiation are invaluable for use in medicine and as a tool for research. However, PSCs show substantial variability both in the pluripotent state and also in their response to differentiation. Thus, although there are many PSCs capable of differentiating into any lineage available, and the tools to generate induced pluripotent stem cells from patients have become routine, not all cell lines behave the same, even when subjected to the exact same culture and differentiation protocols. This variability represents a major hurdle that has held back the otherwise great potential of stem cell models for discovery of new therapeutics and their use in the production of organoids.

At the moment, the use of Wnt inhibitors to improve differential potential, and the use of recombinant human laminin 521 (rhL521) as a coating for plates used for PSC culture to reduce line to line variability, have been described, but these do not work as a standalone solution for the most problematic PSC lines.

There is, therefore, a need for a method to ameliorate existing PSCs to, and maintain them in, a standardised, less variable state, which minimises differences between different lines and allows standardised generation of tissue in vitro for all germ layer derivative cells. This would assist in the establishment of universal protocols and the transfer of technologies between labs, and would mean that PSCs derived from patient samples could readily serve as valuable human disease models, without variability compromising their use.

### SUMMARY OF THE INVENTION

According to a first aspect of the invention there is provided a method of standardising PSCs, the method comprising culturing at least one PSC in cell culture medium supplemented with an inhibitor of a methyltransferase that catalyses the mono-, di- or tri-methylated states of histone H3 at lysine residue 9 (H3K9 MTase), a H3K9 MTase inhibitor.

Although the inventors do not wish to be bound by hypothesis, the above method is believed to reset the chromatin landscape of PSCs, as a result of which standardised PSCs are achieved. The method may further comprise the inclusion of one or more additional small molecules or agents which may also affect the epigenetic landscape. These molecules or agents can be included in the cell culture medium along with H3K9 MTase inhibitor or included as part of a stepwise method in an additional culturing step or steps.

Resetting the chromatin landscape means changing the chromatin landscape of a PSC line so that it is more similar to the chromatin landscape of a PSC line that is naturally competent to differentiate reproducibly and correctly into all 3 germ layers and into, at least, brain-like, kidney-like and intestine-like tissue in particular.

The inventors have found that it is surprisingly possible to standardise PSCs using the method of the first aspect. Standardised PSCs are competent to differentiate into organoids using an unguided method and behave reproducibly and predictably in all germ layer differentiation protocols. Prior to the present invention, PSCs demonstrated variable capacity to differentiate into specific lineages when subjected to the same treatment. Until now, any treatment, including culture and differentiation protocols, needed to be tailored to at least every cell line, if not every clone, thereby limiting large scale application, precluding personalised therapies and preventing their possible application in the clinic and as a research tool. The origins of this variability remain an active field of investigation within the stem cell community. Gene expression and signalling variability, DNA methylation and epigenetic memory, genetic factors and X-inactivation and imprinting have all been investigated to no avail. As described in the Examples, the inventors have now unexpectedly elucidated the origin of this variability at the epigenetic level, and, as a result, have been able to develop a method that overcomes the aforementioned problems.

### DETAILED DESCRIPTION

As used herein, the term “standardising” when used in relation to PSCs has its ordinary meaning in the art, for example standardised refers to having the quality of being comparable to a standard, in this case, so that they behave in the same, predictable way when subjected to the same culture and differentiation protocols.

By the term “stem cell” is meant a cell that can self-renew and differentiate into many different cell types.

The term “pluripotency” refers to a SC that has the potential to differentiate into any of the three germ layers: endoderm, mesoderm or ectoderm, but not into extra-embryonic tissue.

The term “differentiated” when used in relation to SCs means a terminal state, no longer demonstrating features of a stem cell and instead exhibiting specialized features of a final organ cell type such as molecular signature, morphology, and/or function.

The at least one PSC used in the present invention may be an iPSC, a naturally occurring PSC or an ESC. Those skilled in the art will realise that the aforementioned list is not a comprehensive list for use in the present invention. In particular, the PSC used is an iPSC or ESC.

The iPSC used in the present invention could be initially obtained from somatic cells. A somatic cell would be understood by the skilled person to be any cell other than a gamete, germ cell, gametocyte or undifferentiated stem cell. The somatic cell can be obtained from any suitable tissue such as bone marrow, foetal tissue, peripheral blood, umbilical cord blood, pancreas, skin or any organ or tissue. In a particular embodiment, the iPSC is obtained from fibroblasts, blood, adipose-derived cells, neural cells or cells from the intestinal epithelium.

The somatic cell is then treated to render it pluripotent. This induction treatment may involve known methods such as viral transduction, electroporation, or mRNA introduction of reprogramming transcription factors, such as Oct4, Sox2, Klf4, cMyc.

As will now be appreciated, a somatic cell and its reprogramming to generate a PSC are not the subject of this invention. The method of the present invention is used to make already existing PSC lines better, i.e., to improve their differentiation potential (to produce an improved differentiation PSC), so as to render them standardised.

The PSC used in the present invention may be of human, nonhuman primate, rodent, horse, cattle (and other farm animals such as, for example, pig, sheep and chicken), dog, cat or other. This list is not exhaustive and the cell used may be of any mammalian origin. In a particular example, the cell is of human origin.

In a particular embodiment, the PSC are initially obtained from autologous somatic cells, i.e., the cells are autologous. However, the cells can be obtained from heterologous cells.

The PSC to be standardised according to the present invention may be isolated and then maintained in culture and passaged until standardised using methods disclosed for the first time here.

Exemplary media that may be used in a three step method according to the invention are:

### Medium 1

This medium is intended to erase incorrect chromatin changes either leftover from somatic reprogramming of the pluripotent cell line in question or acquired during culture of any pluripotent stem cell line.

The preferred combination of factors with the required biological activities is as follows: H3K9 methyltransferase inhibitor, for example BIX01294, histone deacetylase inhibitor, for example valproic acid (VPA), an SAH hydrolase inhibitor, for example DZNep, inhibitor of histone demethylation, for example tranylcypromine, Dot1L inhibitor, for example, EPZ004777. For specific embodiments of the method this stage could include other small molecules or agents that directly or indirectly affect chromatin and DNA modifications, such as MAPK inhibitor, for example PD0325901, glycogen kinase inhibitor, for example CHIR99021, B-Raf inhibitor for example SB590885, vitamin C or its derivative, for example L-ascorbic acid 2-phosphate. This stage may include culture of cells on coating matrix providing ligands binding integrin a6b1, for example laminin521 or Matrigel.

Although the inventors do not wish to be bound by hypothesis, H3K9 methylation is generally associated with establishment and maintenance of a silent epigenetic state. As cell state commitment proceeds, H3K9 methylation is upregulated at previously open regions of the genome, thus silencing those regions and conferring a different epigenetic and transcriptomic state on the cell.

### Medium 2

This medium is intended to recover cells with reset chromatin landscape.

The preferred combination of factors with the required biological activities is as follows: Wnt inhibitor for example IWP2, selective inhibition of Rho-associated, coiled-coil containing protein kinase (ROCK) for example Y-27632, and ERBB3/4 ligand for example heregulin 1.

In specific embodiments, this stage could include other small molecules or agents that directly or indirectly affect chromatin and DNA modifications, such as MAPK inhibitor, for example PD0325901, glycogen kinase inhibitor, for example CHIR99021, B-Raf inhibitor for example SB590885, vitamin C or its derivative, for example L-ascorbic acid 2-phosphate. This stage may include culture of cells on coating matrix providing ligands binding integrin a6b1, for example laminin521 or Matrigel.

### Medium 3

This medium is intended to re-establish primed state culturing conditions. The preferred combination of factors with the required biological activities is as follows: Wnt inhibitor for example IWP2, selective inhibition of Rho-associated, coiled-coil containing protein kinase (ROCK) for example Y-27632, ERBB3/4 ligand for example heregulin 1, a FGFR1 ligand for example bFGF.

In specific embodiments, this stage could include other small molecules or agents that directly or indirectly affect chromatin and DNA modifications, such as MAPK inhibitor, for example PD0325901, glycogen kinase inhibitor, for example CHIR99021, B-Raf inhibitor for example SB590885, vitamin C or its derivative, for example L-ascorbic acid 2-phosphate. This stage may include culture of cells on coating matrix providing ligands binding integrin a6b1, for example laminin521 or Matrigel.

The PSC may then be cultured in the presence of the H3K9 MTase inhibitor for at least 1, 2, 4, 6, 8, 10, 12, 14, 16, 18, 19 or 20 days. In a particular embodiment, the SC is cultured for 4 days.

The temperature of the culture may be any temperature between 35° C. and 38° C. In an embodiment, the temperature is at least 35° C., 36° C., 37° C. or 38° C.

The media used may be changed at least four times daily, twice daily, daily, every two days or every four days. In a particular embodiment, the media is changed daily.

The PSC may be cultured under hypoxic or normoxic conditions.

The PSC may be cultured in the presence or absence of feeder cells providing extra nutrients, such as mouse embryonic fibroblasts (MEFs), that may be growth arrested through treatment with Mitomycin C or other inhibitors, or gamma irradiation.

The PSC may be cultured in a low percentage of ambient carbon dioxide of 3%, 4%, 5%, 6%, 8%, or 10%. In a particular embodiment, the SC is cultured in 4% carbon dioxide. In addition, the SC may be cultured in the absence of carbon dioxide, but in the presence of other buffering agents such as HEPES.

The PSC may be cultured on Laminin 521 (L521) or any other laminin containing coating such as Matrigel.

The H3K9 MTase to be inhibited may be G9a, SUV39H1, SUV39H2, SETDB1, SETDB2, GLP, PRDM2 or ASH1L. The skilled reader will realise that this list is not exhaustive, but rather illustrative as any H3K9 MTase could be selected to be inhibited when performing the method of the present invention. In a particular embodiment, the H3K9 MTase is G9a.

The H3K9 MTase inhibitor may be BIX01294, BIX01338, UNC0224, UNC0638, UNCO0321 or A-366. Again, the skilled person will realise that this list is not exhaustive, but rather illustrative as any H3K9 MTase inhibitor could be used when performing the method of the present invention. In a particular embodiment, the H3K9 HMTase inhibitor is BIX01294.

In a particular embodiment, the at least one PSC is an iPSC, the H3K9 MTase is G9a and the H3K9 HMTase inhibitor is BIX01294.

The at least one PSC may be cultured in an at least a one, two, three, four or five step cell culture process. In a particular embodiment, the at least one SC is cultured in a three-step cell culture process.

When there is more than one step, a subsequent step comprises culturing at least one PSC from a previous step in a new cell culture medium. In this instance, only one step, in particular the first step, may comprise cell culture medium supplemented with the H3K9 MTase inhibitor, or the H3K9 MTase inhibitor and the inhibitor of histone demethylation. The SC may be maintained in each step for a period of time of at least 1, 2, 3, 4, 5, 6, 7, 8, 9, 10, 11, 12, 13, 14, 15, 16, 17, 18, 19 or 20 days. When there is more than one step, each step may be for the same, or for a different, period of time.

The cell culture medium used in an embodiment of the method may be supplemented with an inhibitor of histone demethylation. This inhibitor of histone demethylation may be tranylcypromine, RN-1, GSK2879, S2101, OG-L002, T-3775440 HCl, Iadademstat (ORY-1001) 2HCl, SP2509, GSK2879552 2HCl, Pulrodemstat (CC-90011) besylate, GSK-LSD1 2HCl or LSD1-C76. Again, the skilled person will realise that this list is not exhaustive, but rather illustrative as any inhibitor of histone demethylation could be used when performing the method of the present invention. In a particular embodiment, the inhibitor of histone demethylation is tranylcypromine.

The disclosed method of standardising PSCs involves inhibition of histone demethylation in the cell being standardised. Tranylcypromine is a nonselective and irreversible monoamine inhibitor (MAOI), which inhibits KDM1A, a dual H3K4 and H3K9 demethylase, and so is able to balance maintenance of H3K4 methylation, which has been shown by the inventors to be beneficial to the standard state of PSCs, and maintenance of H3K9 methylation, which is detrimental.

The cell culture medium used in an embodiment of the method may be supplemented with an inhibitor of histone deacetylases (HDACs). The HADCi may be a HDAC class 1 inhibitor (HDAC1, 2, 3, 8) and may be valproic acid (VPA), Mocetinostat (MGCD0103, MG0103), (−)-Parthenolide, Suberohydroxamic acid, Domatinostat (4SC-202), UF010, Abexinostat (PCI-24781, CRA-024781), Romidepsin (FK228, Depsipeptide, FR 901228, NSC 630176), SR-4370, TC-H 106 (Pimelic Diphenylamide 106), Tacedinaline (CI994, PD-123654, GOE-5549, Acetyldinaline), BRD3308, Entinostat (MS-275, SNDX-275), Suberohydroxamic acid (suberic bishydroxamic acid) and BG45.

The cell culture medium used in an embodiment of the method may be supplement with an inhibitor of histone methyltransferase EZH2 and/or a Dot1L inhibitor. In a particular embodiment, the inhibitor of histone methyltransferase EZH2 may be 3-Deazaneplanocin A (DZNep), GSK 126, GSK 343, JQEZ5, PF 0672630, UNC 1999, UNC2399, ZLD 1039, Tazemetostat (EPZ-6438, E7438), EI1, GSK503, CPI-169, EPZ011989, Lirametostat (CPI-1205), EBI-2511, MS1943, EPZ005687, SHR2554, Tanshindiol C, MC4343, GNA002, Tulmimetostat (CPI-0209), MS8815, MC4355, DM-01, NSC745885, A-395, EPZ005687, (S)-HH2853, YM281 and YM458, and/or the Dot1L inhibitor may be EPZ004777, SGC 0946, Pinometostat (EPZ5676), SYC-522, MU1656, Dot1L-IN-7 (compound 25), Dot1L-IN-4, Dot1L-IN-5, Dot1L-IN-1, Dot1L-IN-1 TFA, EPZ5676 and SGC0946.

The cell culture medium used in any step, or in the single step, if only a one-step procedure is used, may be further supplemented with, or supplemented with, one or more small molecules with the following biological activity:
(i) glycogen kinase inhibitor;
(ii) selective inhibitor of Rho-associated, coiled-coil containing protein kinase (ROCK);
(iii) MAPK inhibitor;
(iv) Wnt inhibitor; and
(v) B-Raf inhibitor;

Although specific factors are disclosed below it should be understood that they can be readily interchanged with known small molecules that provide the same biological activity.

For example, the glycogen kinase inhibitor may be selected from the group consisting of CHIR99021, SB-216763; CHIR 99021 trihydrochloride, BIO-acetoxime, GSK-3β Inhibitor XII, GSK-3 Inhibitor XV, TD114-2, TD114-3, IM12, CHIR98014, AT7519, TWS119, Tideglusib (NP031112), AZD1080, AR-A014418, TDZD-8, LY2090314, WAY-119064, KY19382 (A3051), BRD0705, 1-Alsterpaullone, IM-12 and SB-415286.

The Rho-associated, coiled-coil containing protein kinase (ROCK) inhibitor may be selected from the group consisting of Y-27632, Fasudil, GSK429286A, H-1152 dihydrochloride, Azaindole 1 (TC-S 7001), RKI-1447, Y-39983 HCl, Ripasudil (K-115) hydrochloride dihydrate, Hydroxyfasudil (HA-1100) HCl, Netarsudil (AR-13324) 2HCl, ZINC00881524, thiazovivin, Y-33075, PT-262, CAY10746, Cotosudil, WF-536 and AS1892802.

The MAPK inhibitor may be selected from the group consisting of PD0325901, AZD8330; Trametinib (GSK1120212), U0126, PD184352 (CI-1040), Refametinib (RDEA119), BI-847325, TAK-733, Binimetinib (MEK162), GDC-0623, E6201 (ER-806201), PD 198306, RO4987655, AZD8330 (ARRY-424704), Refametinib (BAY 869766; RDEA119), MAP855, SL327, Selumetinib (AZD6244), Cobimetinib, Trametinib (GSK1120212; JTP-74057), Pimasertib (AS703026), PD318088, Avutometinib, U0126, PD-334581, PD98059, U0124, PD184161, EBI-1051, Zapnometinib (PD0184264), Temuterkib (LY3214996) and Rineterkib.

The Wnt inhibitor may be selected from the group consisting of IWP-2, WNT-C59, XAV-939, CCT251545, IWP 12, IWP L6, Wnt-C59, IWR-1, LGK974 (WNT974), IWP-3, TC-E 5001, IWP-4, IWP-01, JW67, OM-153, YW1128, KY02111, GNF-6231 and DK419.

The B-raf inhibitor may be selected from the group consisting of SB590885, Sorafenib, Vemurafenib (PLX4032), PLX-4720, Dabrafenib (GSK2118436), Regorafenib (BAY 73-4506), RAF265 (CHIR-265), TAK-632, Agerafenib (RXDX-105), GNE-9815, L-779450, B-Raf inhibitor 1 (Compound 13) dihydrochloride, Belvarafenib (HM95573), B-Raf IN 1, AZ304, PLX8394, RAF709, Lifirafenib (BGB-283), CCT196969, LY3009120, Avutometinib, GDC0879, Tinlorafenib (PF-07284890), SHR902275, Everafenib, AZ304, GDC-0879, HG6-64-1, LUT014a and CFT1946.

In a particular embodiment, the cell culture medium or media is/are further supplemented with, or supplemented with at least one of CHIR99021, Y-27632, PD0325901, IWP-2, and SB590885.

The cell culture medium used in the present invention may also be further supplemented with one or more of the following:
(i) N2 supplement;
(ii) B27 supplement;
(iii) GlutaMax;
(iv) MEM Nonessential amino acids;
(v) penicillin-streptomycin;
(vi) L-Ascorbic acid 2-phosphate; and
(vii) heregulinβ-1 (HRG).

Wherein when the at least one SC is cultured in at least two, three, four or five step cell culture process, the cell culture medium used in the last step, may be further supplemented with basic Fibroblast Growth Factor (bFGF) and/or AlbuMax-II supplement.

When the at least one SC is cultured in a three step procedure:
(i) the cell culture medium used in the first step may comprise N2 supplement, B27 supplement, GlutaMax, Nonessential amino acids, penicillin-streptomycin, L-Ascorbic acid 2-phosphate, HRG, CHIR99021, Y-27632, PD0325901, IWP-2, SB590885, VPA, tranylcypromine, DZNep, EPZ004777 and BIX01294;
(ii) the cell culture medium used in the second step may comprise N2 supplement, B27 supplement, GlutaMax, Nonessential amino acids, penicillin-streptomycin, L-Ascorbic acid 2-phosphate, HRG, CHIR99021, Y-27632, PD0325901, IWP-2, SB590885; and
(iii) the cell culture medium used in the third step may comprise N2 supplement, B27 supplement, GlutaMax, Nonessential amino acids, penicillin-streptomycin, L-Ascorbic acid 2-phosphate, HRG, CHIR99021, Y-27632, PD0325901, IWP-2, SB590885, bFGF and AlbuMax-II supplement.

An exemplary protocol for performing the method of the invention is as set out below: On Day 0 the SC cells were split as normal for maintenance (EDTA clump method) on Matrigel or recombinant human laminin 521 (L521) coated plates in their usual culture medium.

Then, on days 1-4/5, the cells were fed cells with Medium 1 (Knockout DMEM with 1% N2 supplement, 2% B27 supplement, 1% Glutamax, 1% Nonessential amino acid, 1% Penicillin-Streptomycin, 50 μg ml⁻¹ L-Ascorbic acid 2-phosphate, 20 ng ml⁻¹ HRG, CHIR99021 (1 μM), Y-27632 (10 μM), PD0325901 (1 μM), IWP-2 (2 μM), SB590885 (0.5 μM), VPA (500 μM), tranylcypromine (5 μM), DZNep (0.05 μM) (may not be included), EPZ004777 (5 μM), BIX01294 (1 μM)), cultured under normoxia with daily medium changes.

Cells were cultured at 37° C., 5% CO₂ at all stages.

From Day 5 or 6 onwards, daily feeds were begun with Medium 2 (Knockout DMEM with 1% N2 supplement, 2% B27 supplement, 1% Glutamax, 1% Nonessential amino acid, 1% Penicillin-Streptomycin, 50 μg ml⁻¹ L-Ascorbic acid 2-phosphate, 20 ng ml⁻¹ HRG, CHIR99021 (1 μM), Y-27632 (10 μM), PD0325901 (1 μM), IWP-2 (2 μM), SB590885 (0.5 μM)). If the cells appeared overgrown at this stage, they were split 1:1 or 1:2 onto a fresh Matrigel or L521 coated plate. The cells were continued to be fed until well defined large areas reminiscent of primed colonies appeared (usually between 5-10 days, often longer). Once these appeared, the cells were split with Accutase onto L521 coated plates into Medium 3 ((Knockout DMEM with 1% N2 supplement, 2% B27 supplement, 1% Glutamax, 1% Nonessential amino acid, 1% Penicillin-Streptomycin, 50 μg ml⁻¹ L-Ascorbic acid 2-phosphate, 20 ng ml⁻¹ HRG, CHIR99021 (1 μM), Y-27632 (10 μM), PD0325901 (0.5 M), IWP-2 (2 μM), SB590885 (0.5 μM), bFGF (100 ng ml⁻¹), AlbuMax-II (2 mg/ml)) (feeder free conditions, medium does not contain bME).

To split the cells, they were washed once with room temperature PBS without calcium and magnesium and then incubated with 0.5 ml Accutase for 5 min at 37° C. The Accutase was then neutralized with 5 ml Knockout DMEM with Y-27632 (10 μM) and cells pelleted at 200 rcf for 4 min, after which the cell pellet was re-suspended in 2 ml of Medium 3 and transferred onto a fresh L521-coated plate.

The cells were then fed daily until compact colonies with typical primed PSC morphology appeared (3-8 days, though precise timing depends on morphology). Daily feeding was continued until colonies were big enough for manual pick up (usually another 4-6 days). Colonies were then picked manually and seeded in Matrigel or L521 coated 24 well plate in Stemflex with Y-27632 (10 μM). The day after, the medium was changed to Stemflex with 2-3 μM IWP2. These clones represent successfully standardized PSCs and could be cultured, expanded and differentiated as normal providing that they are cultured with 2-3 μM IWP2.

The concentration of the N2 supplement, the B27 supplement, GlutaMaxa and NEAA may be at least 0.5%, 1%, 1.5%, 2%, 2.5%, 3%, 3.5%, 4%, 4.5% or 5%.

The concentration of the penicillin-streptomycin may be at least 0.1%, 0.2%, 0.3%. 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2%.

The concentration of Vc2p may be at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 μg ml⁻¹.

The concentration of the HRG may be at least 2, 3, 4, 5, 10, 15, 20, 25, 30, 35, 40, 45, 50, 55, 60, 65, 70, 75, 80, 85, 90, 95 or 100 ng ml⁻¹.

The concentration of the glycogen kinase inhibitor (for example CHIR99021) may be at least 0.1, 0.2, 0.3, 0.4, 0.5, 1, 1.5, 2, 2.5, 3 μM.

The concentration of the ROCK inhibitor (for example Y-27632) may be at least 1, 2, 3, 4, 5, 6, 7, 8, 9, 10, 11, 12, 13, 14, 15, 16, 17, 18, 19, 20 μM.

The concentration of the MAPK inhibitor (for example PD0325901) may be at least 0.3, 0.4, 0.5. 0.6, 0.7, 0.8, 0.9, 1, 1.1, 1.2, 1.3, 1.4, 1.5, 1.6, 1.7, 1.8, 1.9 or 2 μM.

The concentration of the Wnt inhibitor (for example IWP-2) may be at least 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5 M.

The concentration of the B-Raf inhibitor (for example SB590885) may be at least 0.1, 0.2, 0.3, 0.4, 0.5, 1, 1.5, 2 μM.

The concentration of the histone deacetylase inhibitor (for example VPA) may be at least 100, 110, 115, 120, 125, 130, 135, 140, 145, 150, 155, 160, 165, 170, 175, 180, 185, 190, 195, 200, 250, 300, 350, 400, 450, 500, 550, 600, 650, 700, 750, 800, 850, 900, 950 or 1000 μM.

The concentration of inhibitor of histone demethylation (for example tranylcypromine) may be at least 2, 2.5, 3, 3.5, 4, 4.5 or 5 μM.

The concentration of inhibitor of histone methyltransferase (for example DZNep) may be at least 0.01, 0.02, 0.03, 0.04, 0.05, 0.06, 0.07, 0.08, 0.09 or 0.1 μM.

The concentration of Dot1L inhibitor (for example EPZ004777) may be at least 1, 2, 3, 4, 5, 6, 7, 8, 9 or 10 μM.

The concentration of H3K9 MTase inhibitor (for example BIX01294) may be at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 μM.

The concentration of bFGF may be at least 10, 20, 30, 40, 50, 60, 70, 80, 90, 100, 110, 120, 130, 140, 150, 160, 170, 180, 190, 200 ng/ml.

The concentration of AlbuMax-II may be at least 0.2, 0.3, 0.4, 0.5, 0.6, 0.7, 0.8, 0.9, 1, 2, 3, 4, 5, 10, 15, 20 mg/ml.

In a second aspect, there is provided a cell obtained or obtainable by the method according to the first aspect. The cell may be ectoderm, mesoderm or endoderm competent.

In a third aspect, there is provided use of the cell according to the second aspect, in the development of a organoid, as a tool for research, as a model for disease or in the discovery of a therapeutic. The organoid may be a cerebral, renal or digestive tract organoid.

Previously available human drug screening models relied on the patient's samples or immortalised, tumor-derived cell lines. While a patient's cells directly model the effects of a drug on humans, their availability and capacity for expansion are limited and finite compared to in vitro derived cell lines. The latter, however, may contain genetic and metabolic abnormalities due to their derivation, and thus often do not represent a realistic or ideal drug model. These drawbacks restrict the capacity of these models to faithfully simulate disease. By comparison, the cells of the present invention can sidestep these limitations and thus provide a powerful and versatile tool for disease therapy as well as basic research.

In a fourth aspect, there is provided the cell according to the second aspect, for use in therapy or diagnosis.

It is important to note that before its use, the cell may have undergone differentiation, so that it is the cell's differentiated progeny that are used. This also applies to subsequent uses in other aspects of the present invention as disclosed below.

The present invention allows for the further study and development of stem cell technologies, including but not limited to, prophylactic or therapeutic uses. For example, in some embodiments, cells of the invention are introduced into individuals in need thereof, including but not limited to, in need of regeneration of an organ, tissue or cell type. In some embodiments, the cells are originally obtained in biopsy from an individual, induced into pluripotency, standardised as disclosed herein, induced to differentiate and then transplanted back into the individual. The cells may be genetically modified prior to their introduction.

The cells generated according to the method of the invention may be subsequently induced to form, for example hematopoietic cells, neural cells, pancreatic cells, kidney cells, hepatocytes, gastrointestinal tract cells, lung cells, cardiovascular cells, retinal cells, or other types of cells. A variety of protocols are known for inducing PSC into desired cell types.

The cells of the present invention may be induced to form cerebral organoids-artificially grown, in vitro, miniature organs resembling the brain (Lancaster et al. Nature 2013.

Administration of cells described herein is by any of the routes normally used for introducing pharmaceuticals. The cells may be combined with a suitable pharmaceutically acceptable carrier, which may be determined in part by the particular method of administration. Formulations suitable for administration include aqueous and non-aqueous solutions, isotonic sterile solutions, which may contain antioxidants, buffers, bacteriostats and solutes that render the formulation isotonic, and aqueous and no-aqueous sterile suspensions that can include suspending agents, solubilizers, thickening agents, stabilizers and preservatives. In the practice of this invention, the cells may be administered, for example, orally, nasally, topically, intravenously, intraperitoneally, intrathecally, or into the eye. The formulations can be presented in unit-dose or multidose sealed containers, such as ampules and vials.

The dose to be administered to a patient should be sufficient to induce a beneficial response over time. The optimal dose level for any patient will depend on a variety of factors including the age and weight of the patient. Administration can be accomplished via single or divided dose/s.

In a fifth aspect, there is provided a cell culture medium as used in the method according to the first aspect.

In some embodiments, the medium does not comprise cells. In some embodiments, the medium further comprises cells, i.e., at least one SC.

In a sixth aspect, there is provided a kit comprising a cell culture medium according to the fifth aspect.

In some embodiments, the kits further comprise cells, i.e., at least one SC.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### BRIEF DESCRIPTION OF THE DRAWINGS

FIG. 1.

A. Outline of the standard protocol of differentiation of cerebral organoids with Stemdiff Cerebral Organoid Kit (Stemcell Technologies).

B. Top panel—demonstration of variability in the differentiation outcome between different cell lines (H9—very good differentiation, H1—good differentiation, Burb 1—bad differentiation), Bottom panel—demonstration of variability in the differentiation outcome between different batches of the same cell line (H9, left—very good differentiation, middle—good differentiation, right—bad differentiation).

C. Immunofluorescent staining of sections of good differentiators (top panel) and bad differentiators (bottom panel). SOX2 (magenta) labels neural progenitors, TBR2 (green) labels intermediate progenitor specific to dorsal cortex, DAPI (blue) stains all cell nuclei.

FIG. 2.

A. Cell culture on micropatterned laminin 521 droplets and organoids generated from cells cultured on micropatterns, SOX2 (magenta) labels neural progenitors, TBR2 (green) labels intermediate progenitor specific to dorsal cortex, DAPI (blue) stains all cell nuclei.

B. Organoids from cell lines grown on L521 with FGF2 DISCs, SOX2 (magenta) labels neural progenitors, SOX10 (green) labels neural crest, ECAD (white) labels non-neural ectoderm, DAPI (blue) stains all cell nuclei.

C. Organoids from cells grown on L521 with DISCS or with 10 ng/ml NRG1 or with NRG and SRC inhibitor used for EB generation.

D. Organoids from cells grown on L521 with NRG1 and reduced glutathione (GLU) and SRC or with NRG and SRC inhibitor used for EB generation.

E. Organoids from cells grown in Stemflex with 2 μM IWP2 for over 8 passages. F. Organoids from cells grown with 2 μM IWP2 and Ebs generated with IWP2 without (left) or with PI3K inhibitor LY294002 (right), NCAD (green) labels broadly neural cells.

G. Fiaj 1 organoids d9 with DKK1 or without added at the point of Matrigel embedding (d7 of the protocol), fixed after 8h.

FIG. 3.

A. Kolf2 cells (an organoid non-competent batch) cultured in Rset medium for 24 and 72 h show correct morphology for human naïve-like stem cells.

B. Organoids at day 9 made from directly from cells cultured Rset, a typical compact and round EB doesn't form, instead massive cell death is observed.

C. Organoids made from kolf2 cells cultured in Rset followed by 2 days in primed conditions in Stemflex. Organoids display elongated, thin epithelia with morphology different to typical neural epithelia expected at this stage and large areas of dark, disorganized tissue.

FIG. 4.

Similarity tree between peaks of chromatin accessibility of a panel of induced pluripotent stem cells and embryonic stem cells obtained from ATACseq. Bad differentiators HUES8, fiaj 1 and sojd3 form one cluster, whereas good differentiators H1, kolf2 (organoid competent), hehd1 and H9 form another cluster. Interestingly, burb 1 cluster with good differentiators, which could be an explanation as to why treatment only with IWP2 alone was able to restore organoid competency in this line.

FIG. 5.

A. Organoids generated from fiaj 1 cells that had undergone attempted reprogramming following the Guan et al. 2022 protocol from Stage IV media onwards. Organoids embedded in Matrigel (left) do not show neuroepithelial buds and appear dark without obvious structure. Organoids not embedded in Matrigel (right) show irregular shape with local elongated, slightly cleared structures, but not similar to neural epithelia.

B. Organoids from cells that had undergone standardisation using the method of the present invention at day 10 of cerebral organoid protocol show correct morphology for their stage with plentiful, well-formed and cleared epithelial buds.

C. Immunofluorescent analysis of organoids generated from standardised fiaj1 and sojd3 cells generated with the method of the present invention at day 10 and 20. SOX2 (magenta) labels neural progenitors, TBR2 (green) labels intermediate progenitor specific to dorsal cortex, DAPI (blue) stains all cell nuclei, SOX10 (green) labels neural crest, ECAD (white) labels non-neural ectoderm, HUCD (white) labels neurons. Leftmost panel shows typical morphology of organoids generated with the same protocol at day 20 from the cell lines before standardisation.

FIG. 6.

A. and B. The hehd1 cells before treatment (black circles) are already fully competent (good differentiators). Once the other cells lines, which are not competent (bad differentiators: grey circle, white circle with black outline and grey circle with black outline), are treated with the method of the invention and become competent (grey triangle, white triangle with black outline and grey triangle with black outline) they cluster with (are more similar to) the fully competent hehd1 cells (both the black circles and black triangles), which are fully competent both before (black circles) and after (black triangles) treatment with the method of the invention. The dendrograms illustrate this even better.

A. Shows that the chromatin accessibility is the same for hehd1 cells both before (black circles) and after treatment (black triangles), and for burb1, fiaj1, and sojd3 cells after treatment (grey triangle, white triangle with black outline and grey triangle with black outline). The outlined datapoints correspond to the cell lines that are good differentiators. This demonstrates that chromatin accessibility becomes standardized with the treatment.

B. Shows that the RNA expression profile is the same for hehd1 cells both before (black circles) and after treatment (black triangle), and for burb1, fiaj1, and sojd3 cells after treatment (grey triangle, white triangle with black outline and grey triangle with black outline). Data for untreated H9 hES cells (white circle with grey outline) are included for comparison to a naturally competent PSC line and serve as the benchmark for competent cells.

C. Genes UTF1 and GBX2 are more highly expressed in non-competent cells (bad differentiators). Histone 3 lysine 27 (H3K27) methylation inhibits the expression of genes, whereas histone 3 lysine 4 (H3K4) methylation activates expression of genes. This panel shows 4 sets of parallel traces of RNA expression (top), H3K27 methylation (middle) and H3K4 methylation (bottom) for the genomic regions where genes UTF1 and GBX2 are located. The top 3 traces in each set for each gene represent data for untreated cell line fiaj1 (parental) and the bottom 3 traces in each set for each gene represent data for treated cells (CHR). Parental cells show high expression of both UTF1 and GBX2 genes and show low level of inhibitory H3K27 methylation and high level of activating H3K4 methylation. Treated cells show the reverse trend of low expression of both UTF1 And GBX2 genes and show high level of inhibitory H3K27 methylation and low level of activating H3K4 methylation. This demonstrates that the treatment works at the chromatin level and resets the chromatin modifications.

FIG. 7.

A. Shows that the stem cell markers OCT4, SOX2 and NANOG are expressed equally well in all four cell types after treatment with the method of the invention. This demonstrates that the cells are still pluripotent stem cells after treatment.

B. Shows kidney organoid development with magenta and green staining showing generation of epithelial kidney structures. There are four pairs of representative images of organoids generated from parental (untreated) and treated (CHR) cell lines. Hehd1 cells can generate epithelial structures before and after treatment, whereas burb1, fiaj 1 and sojd3 only generate epithelial kidney structures after treatment. Kidneys develop from mesoderm germ layer and the ability of cells prepared according to the method of the invention to generate kidney organoids demonstrates their pluripotency and ability to differentiate into germ layers other than just ectoderm.

C. Quantification of the data from panel B.

D. Cerebral organoids generated from an additional cell line, letw5, treated using the method of the invention. In the top panel there are two example images of brain organoids at day 20 generated from untreated cells (parental). In the bottom panel there are two example images of brain organoids generated from treated cells. Organoids from treated cells show more compact defined borders, lack cysts and large areas of disorganized tissue, and show peripheral clearing where early neural tissue starts to develop.

### EXAMPLES

The invention will now be described by way of illustration only in the following examples.

The inventors have shown that their method of chromatin resetting produces standardised SCs that respond in a standard and predictable way to differentiation and resulted in accurate differentiation of even the most recalcitrant differentiators. Their method equally applies to both naturally occurring PSC and iPSC, and to ESC that have never undergone reprogramming. Moreover, when subjected to a cerebral organoid protocol, their standardised PSCs are readily able to form brain organoids showing correct morphology for their stage of development. In addition, the inventors demonstrate that the standardised PSCs express pluripotency markers SOX2, OCT4 and NANOG and are able to differentiate to derivatives of mesendoderm such as kidney organoids.

Brain organoids may result when PSCs undergo neural differentiation. They can be used to model many aspects of early brain development, defects associated with neurodevelopmental and neurological disorders. Ultimately, therefore, organoids may aid in the discovery of new therapeutics for the aforementioned types of diseases.

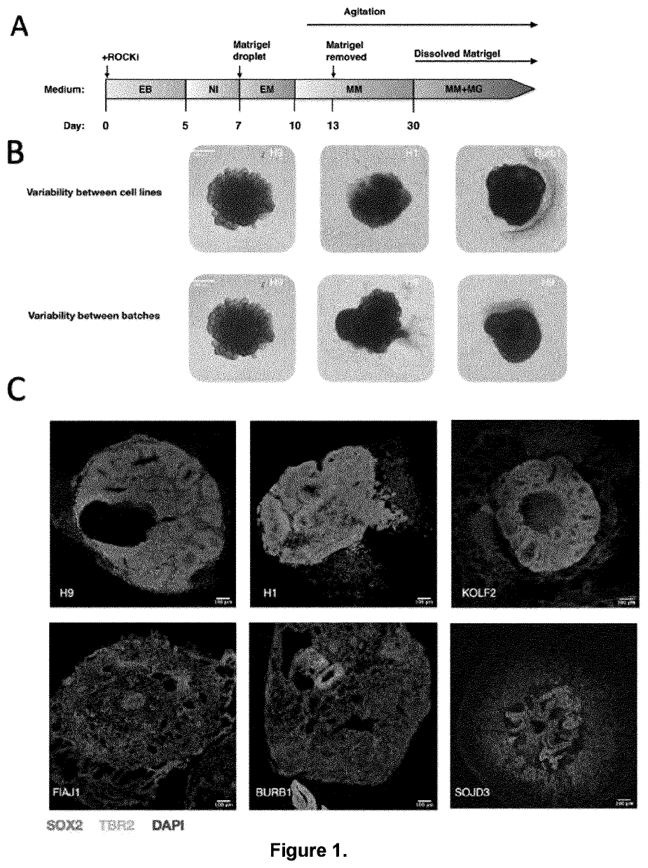

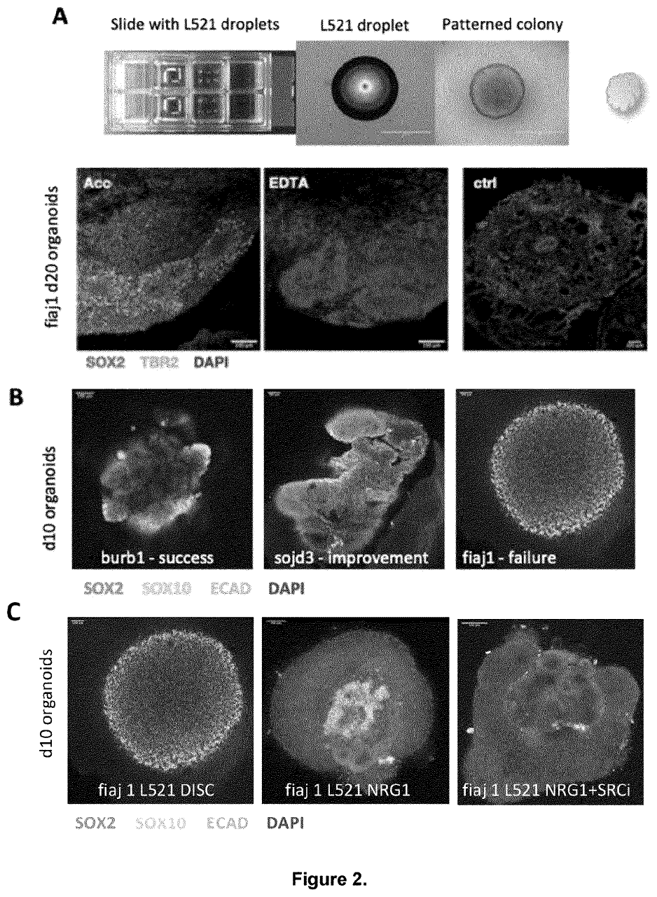

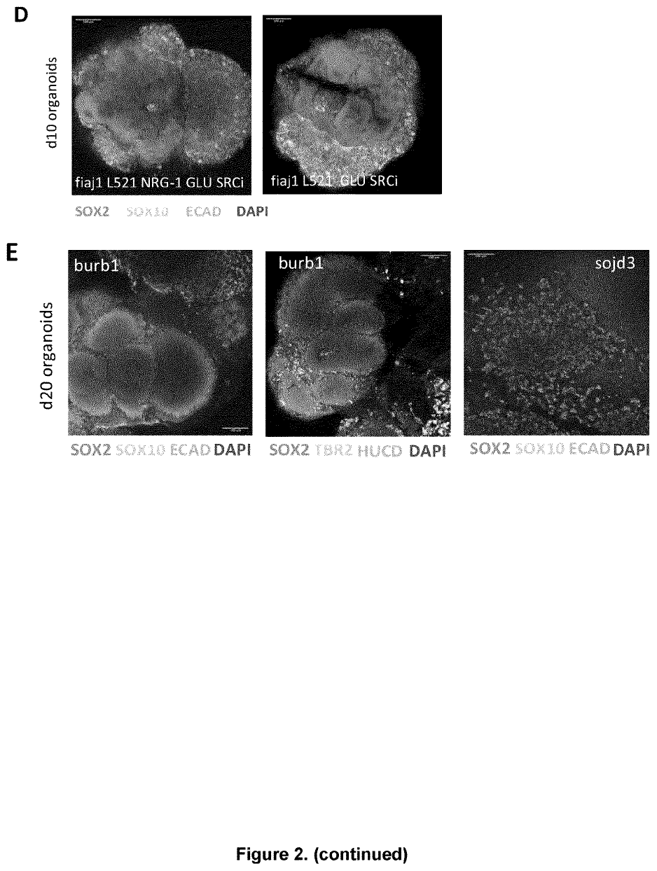

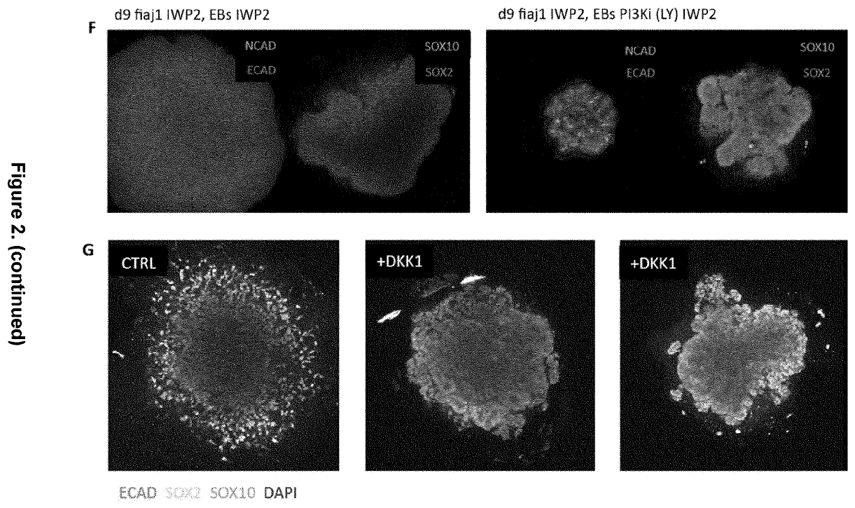

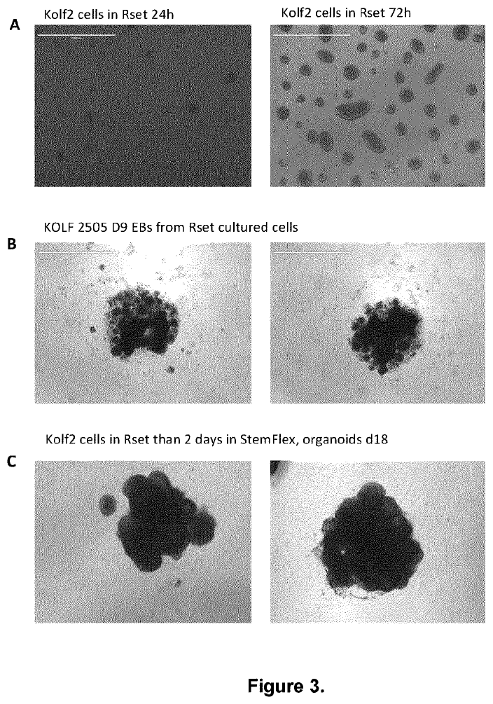

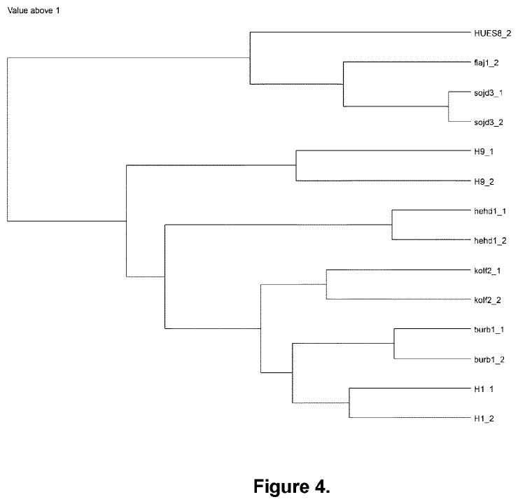

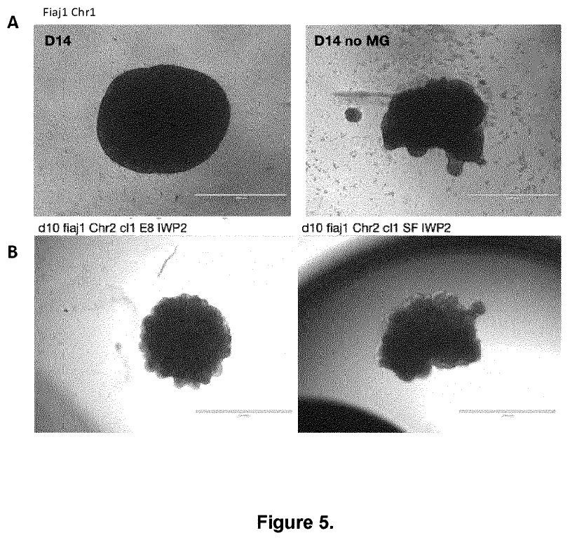

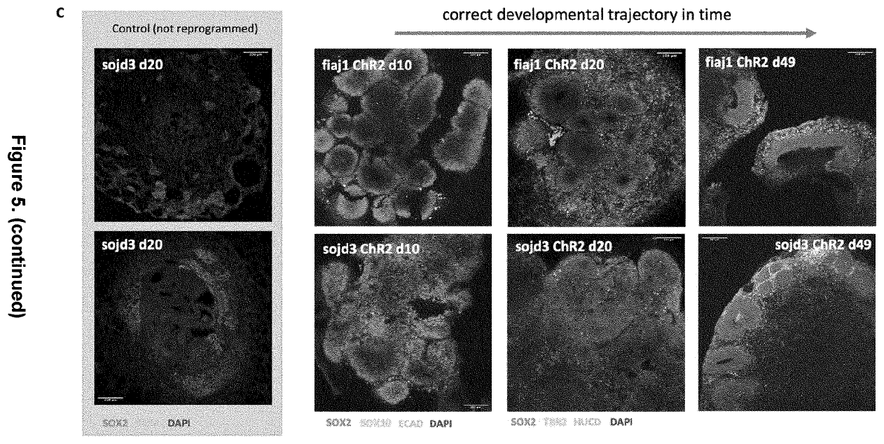

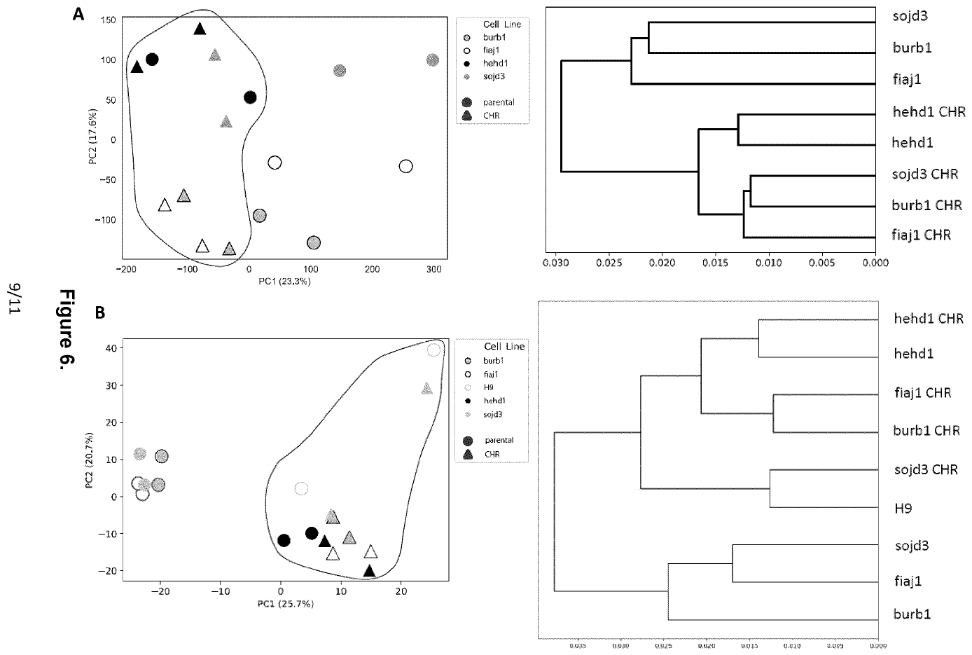

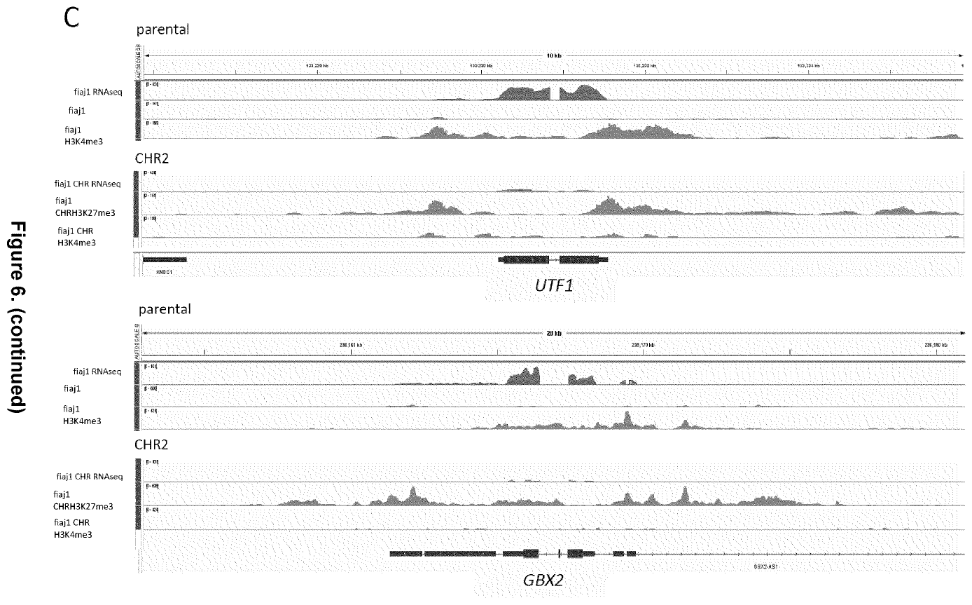

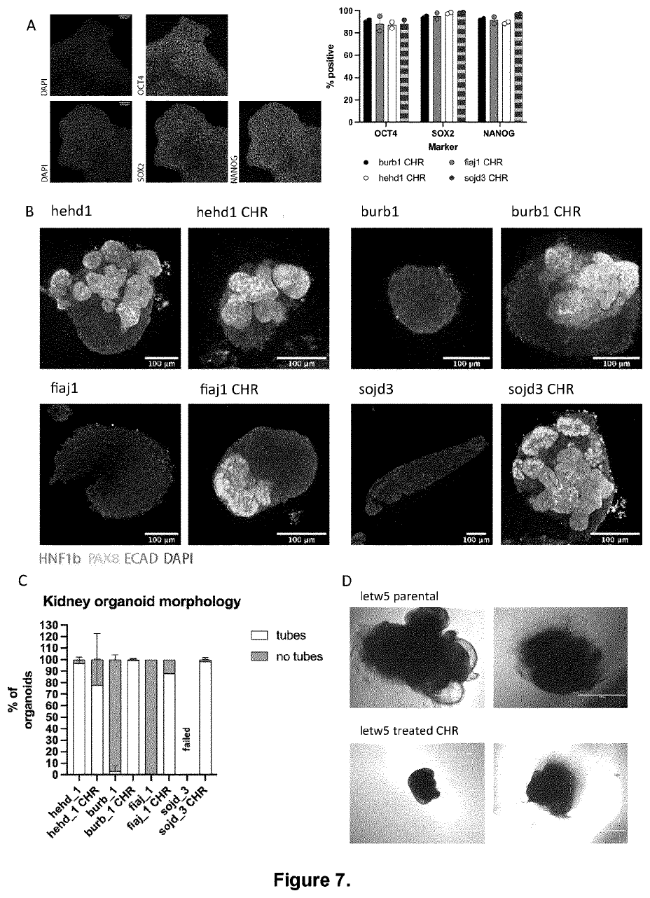

The so called “unguided protocol” relies on the natural propensity of PSCs to differentiate to neural fate and specifically forebrain when not exposed to any growth factors. The inventors cultured PSCs in Stemflex (Thermo) medium on plates coated with Matrigel growth factor reduced (Corning) and for generation of brain organoids they used STEMdiff Cerebral Organoid Kit (Stemcell Technologies). Despite these highly standardised conditions (FIG. 1A), the outcome of the protocol (i.e. the identity and quality of the tissues produced) can vary depending on the cell line that was used in the protocol or even between different batches of the same line (FIGS. 1 B and C). This makes producing good quality organoids from different lines difficult and unpredictable. To address this variability that plagues not only the neural organoid field but also others that rely on 2D and 3D differentiation to a variety of cell types, the inventors investigated the potential sources of this variability in PSCs and potential treatments to address this problem or, in other words, to standardise the performance of PSCs during differentiation.

### Example 1—Known Treatments Tested

There is a huge body of literature describing potential sources of PSC variability and a similar number of publications about ways to address this in a particular context, for example to increase efficiency of differentiation towards a certain tissue type. One approach in the inventors' study was to conduct a thorough literature search and try to adapt already published interventions or test combinations thereof at the stage of PSC maintenance stage. The inventors reasoned that since in the most undifferentiated state, PSCs are amenable to neural differentiation upon pluripotency exit, by modifying culture conditions they could promote such basal or standard state in all cell lines.

To do so, the inventors selected a small panel of PSC lines that did not readily differentiate into neural tissue and tested published treatment and their combinations on these lines. Because neural differentiation represents the default program upon pluripotency exit, the ability of a cell line to undergo such unguided differentiation is also a good indication of their pluripotent state. The other approach was to in depth characterise PSC lines that either produce good or bad organoids by proteomics and phosphoproteomics, analysis of already available bulk RNAseq data to learn about their transcriptome, and by ATACseq to learn about open chromatin patterns. This multifaceted approach would then help the inventors build a better picture of what cellular processes distinguish good and bad differentiators and inform potential new treatments. Based on the combination of information gathered from both approaches, a number of treatments were chosen to test on the bad differentiators. In Table 1, the inventors have summarised all the treatments tested, and in FIG. 2 are the treatments that were either somewhat successful, or only successful in some cell lines (and therefore not useful since they were not universal). Unfortunately, none of the previously described approaches, nor their subtle modifications, were sufficient to correct deficient differentiation in all cell lines.

TABLE 1

| Treatment | Rationale and references | Effect |
|---|---|---|
| culture on L521 | L521 engages integrin a6 and prevents | improved organoid generation |
| | overactivation of FAK that leads to loss | in burb1, better if combined |
| | of pluripotency (Villa-Diaz et al. 2016), | with L521 |
| | cells cultured on laminin show fewer | |
| | large focal adhesions and fewer actin | |
| | stress fibers than when cultured on | |
| | vitronectin, proteomics results showed | |
| | more dynamic actin cytoskeleton in bad | |
| | differentiators | |
| inhibition of SRC at | Direct inhibition of FAK was avoided | Worse organoid differentiation |
| basal culture (CGP- | due to literature evidence that some | outcome, differentiation of PS |
| 77675) | activity of FAK is needed for survival | cells with prolonged exposure |
| | and pluripotency maintenance (Vitillo et | |
| | al. 2016) and that FAK plays a role in | |
| | pro-survival signalling through IGF1R | |
| | (Godoy-Parejo et al. 2019) but bad | |
| | differentiators show higher FAK | |
| | signalling that is improved with L521 | |
| | culture therefore FAK-SRC signalling | |
| | looked like a promising target | |
| inhibition of SRC at | chemical inhibition of SRC family | no improvement alone, |
| organoid generation | kinases or knockdown of SRC improve | improves outcome when used |
| (WH-4-023) | differentiation towards all germ layers | alongside with HRG |
| | (Chetty et al. 2015), inhibition or SRC | |
| | caused epithelial differentiation (Lian et | |
| | al. 2013), bad differentiators show higher | |
| | FAK signalling that is improved with | |
| | L521 culture therefore FAK-SRC | |
| | signalling looked like a promising target | |
| FGF2 DISCS- | FGF2 is necessary to maintain | in cells cultured on L521 |
| sustained FGF2 | pluripotency and its withdrawal leads to | improved organoid |
| release, 3 feeds a | differentiation (Vallier, Alexander, and | differentiation in burb 1, |
| week | Pedersen 2005), addition of TGF family | somewhat improved |
| | ligands improves telencephalic organoid | differentiation in sojd3 but |
| | differentiation (Watanabe et al. 2022), | with bad morphology, no |
| | cocktails of TGFbeta ligands increases | difference in fiaj1 |
| | ERK phosphorylation, same as FGF, | |
| | sustained release FGF2 from DISCs | |
| | improves expression of stem cell | |
| | markers, increases stem cell numbers | |
| | and decreases spontaneous | |
| | differentiation (Lotz et al. 2013) | |
| culture with HRG | bFGF, a growth factor essential for PSC | some improvement in fiaj1, |
| | maintenance, leads to phosphorylation of | better if combined with |
| | ERBB3, a HRG co-receptor (Ding et al. | antioxidants or SRC inhibitor |
| | 2011), self-renewal of PSCs requires | WH-4-023 |
| | ERBB2/ERBB3 activation that can be | |
| | achieved with HRG (Wang et al. 2007), | |
| geometric | culture of PSCs on micropatterns | worked to some extent in fiaj 1 |
| constriction-culture | standardises their response to | (areas of correct differentiation |
| on L521 | gastrulation inducing cues in vitro | present but a small percentage |
| micropatterns | (Warmflash et al. 2014), constrained | of overall organoid volume) |
| | cells in the center of micropatterns are | |
| | insensitive to WNT signals that induce | |
| | gastrulation like process at the colony | |
| | periphery (Martyn, Brivanlou, and | |
| | Siggia 2019) | |
| organoid culture | Noggin/SB431542 facilitate neural | Only using Noggin/SB431542 |
| with dual SMAD | induction in human PSCs (Chambers et | for longer shows any |
| inhibition | al. 2009), short pulse of | improvement, leading to |
| | Noggin/SB431542 used for 1-3 days in | change of the character of the |
| | the first 3 days of organoid protocol | differentiation protocol from |
| | | unguided to guided |
| generation of | addition of PD0325901 (inhibitor of | Some improvement in bad |
| organoids with MEK | MEK and downstream ERK) improves | batch of kolf2, no |
| inhibition | neural ectoderm differentiation from | improvement in fiaj 1 |
| | mouse epiblast stem cells (Yu et al. | |
| | 2017), FGF signalling prevents neural | |
| | induction through ERK activation in | |
| | human PSCs (Greber et al. 2011) | |
| resetting cells (first | hypothesis-resetting cells to naive | cells die at EB stage if EBs |
| to naive by Rset and | conditions could erase aberrant | were generated straight from |
| then re-priming in | epigenetic marks that prevent neural | cells cultured in Rset, when |
| Stemflex) | differentiation, done with Rset protocol | cells were returned to primed |
| | (Stemcell Technologies) | conditions organoids form but |
| | | with no improvement to |
| | | morphology |
| reducing glucose in | high levels of glucose in standard | together with lower insulin |
| culture medium | culture media for PSCs contribute to | cells die |
| | oxidative stress and bias cells towards | |
| | mesoderm and cardiomyocyte | |
| | differentiation (Crespo et al. 2010), | |
| | inventors' proteomics results suggest | |
| | higher oxidative metabolism in bad | |
| | differentiators | |
| eliminating pyruvate | exogenous pyruvate enhances | together with lower insulin |
| from medium | mesodermal differentiation of PSCs | cells die |
| | (Song et al. 2019) | |
| long term culture in | culture in E8 leads to improved directed | no improvement and in some |
| E8 medium | neural differentiation (Cornacchia et al. | cell lines deterioration |
| | 2019) | |
| addition of alpha- | addition of alpha-ketoglutrate improves | Embryoid bodies don't form |
| ketoglutarate to | differentiation of primed pluripotent | due to cell death |
| generate EBs | stem cells though modification of histone | |
| | and DNA methylation (TeSlaa et al. | |
| | 2016), culture in E8 leads to improved | |
| | directed differentiation into neural tissue | |
| | through increasing alpha- | |
| | ketoglutarate/succinate ratio (Cornacchia | |
| | et al. 2019) | |
| pre-treatment with | FGFR1 inhibition for two days before | didn't see improvement, sojd3 |
| FGFR1 inhibitor | organoid generation improves identity | might look worse |
| | and morphology to the same extent as | |
| | culture on feders (Ideno et al. 2022) | |
| antioxidants | high level of oxidative metabolism and | some improvement in fiaj1, |
| (reduced glutathione | ROS bias towards mesoderm | better when combined with |
| and NAC) added to | differentiation (Crespo et al. 2010; Song | HRG or HRG and SRC |
| culture before | et al. 2019), inventors' proteomics | inhibitor WH-4-023 |
| making Ebs and to | results suggest increased mitochondrial | |
| Ebs | oxidative metabolism in bad | |
| | differentiators | |
| inhibition of PI3K | PI3K drives PSC survival but also | addition of PI3K inhibitor at |
| during EB | increases oxidative metabolism (Ren et | the point of EB generation |
| generation, lowering | al. 2020), there is some phenotypic | together with Wnt inhibition |
| insulin in culture | overlap at transcriptomic and proteomic | improve organoid |
| media | level between bad cell lines and cells | differentiation in fiaj1 line |
| | with mutant PIK3CA H1047R allele | |
| | (Madsen et al. 2021) | |
| inhibition of Wnt in | human ES cells with low Wnt activity | only works if applied long |
| culture | are biased towards neuro-ectoderm | term (>8 passages), |
| | and high Wnt towards mesendoderm | improved organoid |
| | in directed differentiation, IWP2 in | generation in burb1 cells, |
| | culture media before differentiation | no improvement in sojd3 |
| | decreases Wnt reporter signal and | |
| | biases towards neuroectoderm | |
| | (Blauwkamp et al. 2012), Wnt | |
| | inhibition prevents spontaneous | |
| | differentiation of hESCs and epiblast | |
| | stem cells and reverts them to pre- | |
| | gastrula state (Kurek et al. 2015) | |
| inhibition of Wnt at | Wnt signalling drives neural crest | DKK-1 or IWP2 added at |
| organoid stage | induction (Ji et al. 2019) and inventors' | embedding stage improve |
| | immunofluorescent staining shows that | organoid morphology |
| | bad differentiators produce neural crest | |
| | rather than brain tissue | |

### Example 2—Development of an Initial Method to Standardise Pluripotent Stem Cells

With failure to discover a set of universal stem cell culture conditions to produce PSCs that respond in a standard, predictable way to differentiation, the inventors hypothesized that the differences between lines are either genetic and cannot be changed, or epigenetic and can be manipulated. Although bad differentiators showed some phenotypic overlap with cells with mutant PIK3CA H1047R allele (Madsen et al. 2021) and certain cell lines used for this study carry pathological mutations (fiaj 1 and sojd3 in BCOR, (Puigdevall et al. 2023), the inventors also had evidence that cells that performed badly in their hands produced good tissues in other labs (sojd3, (Kanton et al. 2019)). This suggested that the variability between cell lines could be epigenetic.

The inventors tested resetting a PSC line to the naïve state and then returning them to primed conditions. Conversion of primed cells to naïve state is associated with massive chromatin remodelling (reviewed by (Sun et al. 2021)) and they speculated it could erase potential epigenetic changes that prevented cerebral differentiation competency.

The inventors used RSeT Medium and conversion to naïve-like cells was shown to be successful in a bad batch of kolf2. However, on return to primed culture conditions these cells did not improve in their ability to generate cerebral organoids, suggesting that a more targeted approach could be needed (FIG. 3).

### Example 3—Derivation of a More Targeted Approach

To establish such an approach, the inventors analysed the epigenetic state of human pluripotent stem cells and the role of epigenetics in development. Human pluripotent stem cells are similar to human embryonic epiblast before gastrulation. During gastrulation Wnt signalling drives acquisition of the primitive streak fate, which is associated with massive chromatin rearrangement, whereas chromatin of the non-gastrulating epiblast stays largely the same and does not undergo large rearrangements during neural differentiation (Argelaguet et al. 2019). The inventors speculated that the bad differentiators might have a higher level of Wnt signalling leading to some chromatin changes that could be reverted by blocking the Wnt signals that initiate and maintain them. Wnt inhibition was previously demonstrated to improve neural differentiation (Blauwkamp et al. 2012) and to prevent primitive streak bias in mouse primed stem cells (Kurek et al. 2015). Wnt inhibition was effective in the case of the burb 1 cell line. Burb 1 cells grown in Stemflex medium with 2-3 μM IWP2 acquired brain organoid competency after over 8 passages, however sojd3 cells did not improve under the same treatment, suggesting that while this may reverse the suboptimal state in some cell lines where the epigenetic state was not too degraded, it was not sufficient in others. This suggested that a stronger approach may be needed, one that works on epigenetic modifying enzymes rather than their upstream regulators.

### Example 4—Derivation of a Stronger More Targeted Approach

In the embryo, gastrulation and formation of the primitive streak result in an irreversible fate change from anterior neural-competent epiblast to cells that can only produce mesoderm and endoderm (Argelaguet et al. 2019). The inventors therefore hypothesized that cell lines that easily differentiate to neural fate have a chromatin state more similar to early epiblast, and the bad differentiators have acquired some features of primitive streak at the epigenetic level, which makes them incompatible with differentiation towards brain tissue. They have indeed shown that the pattern of open chromatin peaks analysed by bulk ATACseq differs between good and bad differentiators (FIG. 4) and they hypothesise that these peaks suggest changes similar to primitive streak identity in bad differentiators.

Since primitive streak cells are developmentally more advanced than early epiblast, the inventors sought a cocktail of epigenetic modifiers that would reverse this developmental progression. In mouse, cells equivalent to early epiblast, so called epiblast like cells (EpiLCs), don't show lineage commitment and display lower levels of repressive histone marks than lineage committed cells, in particular H3K27me3 and H3K9me2, but higher levels of bivalent promoters, decorated by both H3K4me3 and H3K27me3, and high levels of histone acetylation (Kurimoto et al. 2015). Blocking histone deacetylation also helps with resetting human primed cells to naïve cells. Therefore, inhibition of methylation of H3K27 and H3K9, and increasing histone acetylation could possibly promote a lineage uncommitted state.

There is also a body of evidence that inhibition of several epigenetic modifiers improves classic reprogramming of somatic cells to induced pluripotent stem cells with the use of OSKM factors (OCT4, SOX2, KLF4 and MYC). Transient overexpression of the OSKM factors is able to completely remodel somatic chromatin and convert a terminally differentiated cell to a cell that fulfils the criteria of pluripotency (Onder et al. 2012). Simultaneous addition of vitamin C, a co-factor for TET and Jumonji (JMJ) domain-containing proteins, improves histone and DNA demethylation (Blaschke et al. 2013; T. Wang et al. 2011), whereas inhibition of DOTL1, a H3K79 histone methyltransferase, aids with silencing fibroblast-specific genes involved in epithelial to mesenchymal transition, a process that needs to be reversed to obtain iPSCs of epithelial character (Onder et al. 2012). The inventors therefore devised that inclusion of vitamin C and a H3K79 histone methyltransferase could be beneficial in standardising pluripotent stem cells.

A particular obstacle to successful reprograming and complete erasure of the somatic chromatin programme is methylation of H3K9 (Chen et al. 2013; Becker, Nicetto, and Zaret 2016). Even after reprogramming, induced PSCs often display large domains of aberrantly methylated DNA resistant to reprogramming due to the presence of the heterochromatin mark H9K3me3 (Lister et al. 2011). In normal embryonic development H3K9 methylation plays a role in permanent silencing of chromatin to establish both constitutive and facultative heterochromatin and to prevent reactivation of transposable genetic elements (Hawkins et al. 2010; Montavon et al. 2021; Xu et al. 2022). Deposition of H3K9me3 marks on lineage specific promoters after implantation represses the extraembryonic fate in mouse epiblast and vice versa (C. Wang et al. 2018). During gastrulation, increase of H3K9me3 in gene bodies, promoters and termination transcription sites is associated with early endoderm and mesoderm germ layer specification (Nicetto et al. 2019) and in somatic cells H3K9 methylation represses lineage-specific promoters and enhancer elements (Padeken, Methot, and Gasser 2022). Aberrant H3K9 methylation can also arise as an artifact of in vitro cell culture (Zhu et al. 2103; Chen et al. 2013). Inhibition or silencing of enzymes that methylate H3K9 improves somatic cell reprogramming with OSKM factors (Shi et al. 2008; Chen et al. 2013; Song et al. 2022).

Recently, Guan et al. (Guan et al. 2022) developed a multi-step process of reprogramming human foetal or adult somatic cells to induced pluripotent stem cells using chemical/small molecule means, as opposed to the more established method of introducing OSKM factors. This suggests that small molecules alone may be capable of resetting the epigenetic state of cells. However, their method begins with somatic, terminally differentiated cells and involves several intermediates, thus making it not applicable to the inventors' starting material of already pluripotent stem cells. In contrast to Guan et al., the inventors' method targets changes that were most likely acquired after full reprogramming and during subsequent cell culture. In addition, their method also applies to both induced pluripotent stem cell and embryonic stem cells that have been derived from embryos and have never undergone reprogramming.

Indeed, it is important to mention that application of Medium IV and the following steps from Guan et al. protocol did not result in standardised pluripotent stem cells that could generate brain organoids. Although some cell colonies reminiscent of PSCs were isolated and could be propagated, differentiation in the inventors' standard cerebral organoid protocol did not result in good quality organoids but produced disorganised tissue similar to untreated cell lines (FIG. 5A.).

The inventors have developed a new method focused on removal of H3K9 methylation by addition of BIX01294 (1 μM) in Medium 1, an inhibitor of G9a H3K9 methyltransferase, or inhibition of any other H3K9 methyltransferase such as SUV39H1, SUV39H2, SETDB1, SETDB2, GLP, PRDM2 or ASH1L. They have also balanced the concentration of tranylcypromine, which inhibits KDMIA, a dual H3K4 and H3K9 demethylase, to achieve maintenance of methylation on H3K4, which is beneficial to the standard state of pluripotent stem cells, and maintenance of H3K9 methylation, which is detrimental. The method described here does not use b-mercaptoethanol, which can be toxic to cells. Cells are present on L521 (Laminin 521) or other Laminin containing coatings such as Matrigel, rather than on a feeder layer, for ease and reproducibility. The use of L521 is also supported by the evidence that culture on L521 improves pluripotency and reproducibility of human cultures (Albalushi at al. 2018) and by their own findings with patterned L521 colonies. The inventors are currently optimising concentrations of DZNep, an EZH2 inhibitor, especially for application on ES cells, since inhibition of EZH has been shown to bias PSCs towards mesoderm differentiation (Yu et al. 2017). Their method is also followed by maintenance medium—Stemflex with 2-3 μM IWP2—to prevent relapse. Overall, this method was demonstrated to result in accurate differentiation of even the most stubborn bad differentiators (FIG. 5B.). Moreover, further analysis of older organoids using immunofluorescence confirmed the expression of correct markers of dorsal forebrain (TBR2) and correct tissue morphology, in stark contrast to the organoids generated from untreated cell lines (FIG. 5C).

Standardised cell lines obtained using the exemplary protocol above showed the corrected pattern of chromatin opening, transcriptome and histone marks (FIG. 6A. to C.). The standardised cell lines express the three pluripotency markers SOX2, OCT4 and NANOG (FIG. 7A.), and are able to differentiate into mesoderm derivatives with efficiencies equal to or higher than their untreated counterparts (FIGS. 7B. and C.). The method has now also been successfully applied to other cell lines, for example letw5, a line, which until now was associated with poor brain organoid differentiation outcomes (FIG. 7D.) and performed well.

### SUMMARY

The methods described herein provide a new and flexible way to standardise PSCs. Being able to standardise PSCs, as the inventors have done, advantageously allows PSCs to be utilised to their full potential in the generation of all germ layer derivatives. This new technology therefore makes it possible to use PSCs as a model for disease and as a substitute for limited animal models. Such PSC based disease models can be used to understand the mechanics underlying the cellular, molecular and physiological phenotypes of disease, as well as to develop new therapies to attenuate the disorders concerned by screening for potential drugs.

### REFERENCES

Albalushi, Halima, Magdalena Kurek, Leif Karlsson, Luise Landreh, Kristín Rós Kjartansdóttir, Olle Söder, Outi Hovatta, and Jan-Bernd Stukenborg. 2018. ‘Laminin 521 Stabilizes the Pluripotency Expression Pattern of Human Embryonic Stem Cells Initially Derived on Feeder Cells’. Stem Cells International 2018:7127042. https://doi.org/10.1155/2018/7127042.
Argelaguet, Ricard, Stephen J. Clark, Hisham Mohammed, L. Carine Stapel, Christel Krueger, Chantriolnt-Andreas Kapourani, Ivan Imaz-Rosshandler, et al. 2019. ‘Multi-Omics Profiling of Mouse Gastrulation at Single-Cell Resolution’. Nature 576 (7787): 487-91. https://doi.org/10.1038/s41586-019-1825-8.
Becker, Justin S., Dario Nicetto, and Kenneth S. Zaret. 2016. ‘H3K9me3-Dependent Heterochromatin: Barrier to Cell Fate Changes’. Trends in Genetics: TIG 32 (1): 29-41. https://doi.org/10.1016/j.tig.2015.11.001.
Blaschke, Kathryn, Kevin T. Ebata, Mohammad M. Karimi, Jorge A. Zepeda-Martínez, Preeti Goyal, Sahasransu Mahapatra, Angela Tam, et al. 2013. ‘Vitamin C Induces Tet-Dependent DNA Demethylation and a Blastocyst-like State in ES Cells’. Nature 500 (7461): 222-26. https://doi.org/10.1038/nature12362.
Blauwkamp, Timothy A., Shelly Nigam, Reza Ardehali, Irving L. Weissman, and Roel Nusse. 2012. ‘Endogenous Wnt Signalling in Human Embryonic Stem Cells Generates an Equilibrium of Distinct Lineage-Specified Progenitors’. Nature Communications 3:1070. https://doi.org/10.1038/ncomms2064.
Chambers, Stuart M, Christopher A Fasano, Eirini P Papapetrou, Mark Tomishima, Michel Sadelain, and Lorenz Studer. 2009. ‘Highly Efficient Neural Conversion of Human ES and IPS Cells by Dual Inhibition of SMAD Signaling’. Nature Biotechnology 27 (3): 275-80. https://doi.org/10.1038/nbt.1529.
Chen, Jiekai, He Liu, Jing Liu, Jing Qi, Bei Wei, Jiaqi Yang, Hanquan Liang, et al. 2013. ‘H3K9 Methylation Is a Barrier during Somatic Cell Reprogramming into IPSCs’. Nature Genetics 45 (1): 34-42. https://doi.org/10.1038/ng.2491.
Chetty, Sundari, Elise N. Engquist, Elie Mehanna, Kathy O. Lui, Alexander M. Tsankov, and Douglas A. Melton. 2015. ‘A Src Inhibitor Regulates the Cell Cycle of Human Pluripotent Stem Cells and Improves Directed Differentiation’. The Journal of Cell Biology 210 (7): 1257-68. https://doi.org/10.1083/jcb.201502035.
Cornacchia, Daniela, Chao Zhang, Bastian Zimmer, Sun Young Chung, Yujie Fan, Mohamed A. Soliman, Jason Tchieu, et al. 2019. ‘Lipid Deprivation Induces a Stable, Naive-to-Primed Intermediate State of Pluripotency in Human PSCs’. Cell Stem Cell 25 (1): 120-136.e10. https://doi.org/10.1016/j.stem.2019.05.001.
Crespo, Francisco Luna, Veronica R. Sobrado, Laura Gomez, Ana M. Cervera, and Kenneth J. McCreath. 2010. ‘Mitochondrial Reactive Oxygen Species Mediate Cardiomyocyte Formation from Embryonic Stem Cells in High Glucose’. Stem Cells (Dayton, Ohio) 28 (7): 1132-42. https://doi.org/10.1002/stem.441.
Ding, Vanessa M. Y., Paul J. Boersema, Leong Yan Foong, Christian Preisinger, Geoffrey Koh, Subaashini Natarajan, Dong-Yup Lee, et al. 2011. ‘Tyrosine Phosphorylation Profiling in FGF-2 Stimulated Human Embryonic Stem Cells’. PloS One 6 (3): e17538. https://doi.org/10.1371/journal.pone.0017538.
Godoy-Parejo, Carlos, Chunhao Deng, Weiwei Liu, and Guokai Chen. 2019. ‘Insulin Stimulates PI3K/AKT and Cell Adhesion to Promote the Survival of Individualized Human Embryonic Stem Cells’. Stem Cells (Dayton, Ohio) 37 (8): 1030-41. https://doi.org/10.1002/stem.3026.
Greber, Boris, Philippe Coulon, Miao Zhang, Sören Moritz, Stefan Frank, Arnoldo José Müller-Molina, Marcos J. Arauzo-Bravo, Dong Wook Han, Hans-Christian Pape, and Hans R. Schöler. 2011. ‘FGF Signalling Inhibits Neural Induction in Human Embryonic Stem Cells’. The EMBO Journal 30 (24): 4874-84. https://doi.org/10.1038/emboj.2011.407.
Guan, Jingyang, Guan Wang, Jinlin Wang, Zhengyuan Zhang, Yao Fu, Lin Cheng, Gaofan Meng, et al. 2022. ‘Chemical Reprogramming of Human Somatic Cells to Pluripotent Stem Cells’. Nature 605 (7909): 325-31. https://doi.org/10.1038/s41586-022-04593-5.
Hawkins, R. David, Gary C. Hon, Leonard K. Lee, Queminh Ngo, Ryan Lister, Mattia Pelizzola, Lee E. Edsall, et al. 2010. ‘Distinct Epigenomic Landscapes of Pluripotent and Lineage-Committed Human Cells’. Cell Stem Cell 6 (5): 479-91. https://doi.org/10.1016/j.stem.2010.03.018.
Ideno, Hirosato, Kent Imaizumi, Hiroko Shimada, Tsukasa Sanosaka, Akisa Nemoto, Jun Kohyama, and Hideyuki Okano. 2022. ‘Human PSCs Determine the Competency of Cerebral Organoid Differentiation via FGF Signaling and Epigenetic Mechanisms’. IScience 25 (10): 105140. https://doi.org/10.1016/j.isci.2022.105140.
Ji, Yu, Hongyan Hao, Kurt Reynolds, Moira McMahon, and Chengji J. Zhou. 2019. ‘Wnt Signaling in Neural Crest Ontogenesis and Oncogenesis’. Cells 8 (10): 1173. https://doi.org/10.3390/cells8101173.Kanton, Sabina, Michael James Boyle, Zhisong He, Malgorzata Santel, Anne Weigert, Fátima Sanchís-Calleja, Patricia Guijarro, et al. 2019. ‘Organoid Single-Cell Genomic Atlas Uncovers Human-Specific Features of Brain Development’. Nature 574 (7778): 418-22. https://doi.org/10.1038/s41586-019-1654-9.
Kurek, Dorota, Alex Neagu, Melodi Tastemel, Nesrin Tüysüz, Johannes Lehmann, Harmen J. G. van de Werken, Sjaak Philipsen, et al. 2015. ‘Endogenous WNT Signals Mediate BMP-Induced and Spontaneous Differentiation of Epiblast Stem Cells and Human Embryonic Stem Cells’. Stem Cell Reports 4 (1): 114-28. https://doi.org/10.1016/j.stemcr.2014.11.007.

Kurimoto, Kazuki, Yukihiro Yabuta, Katsuhiko Hayashi, Hiroshi Ohta, Hiroshi Kiyonari, Tadahiro Mitani, Yoshinobu Moritoki, et al. 2015. ‘Quantitative Dynamics of Chromatin Remodeling during Germ Cell Specification from Mouse Embryonic Stem Cells’. Cell Stem Cell 16 (5): 517-32. https://doi.org/10.1016/j.stem.2015.03.002.

Lian, Xiaojun, Joshua Selekman, Xiaoping Bao, Cheston Hsiao, Kexian Zhu, and Sean P. Palecek. 2013. ‘A Small Molecule Inhibitor of SRC Family Kinases Promotes Simple Epithelial Differentiation of Human Pluripotent Stem Cells’. PloS One 8 (3): e60016. https://doi.org/10.1371/journal.pone.0060016.
Lister, Ryan, Mattia Pelizzola, Yasuyuki S. Kida, R. David Hawkins, Joseph R. Nery, Gary Hon, Jessica Antosiewicz-Bourget, et al. 2011. ‘Hotspots of Aberrant Epigenomic Reprogramming in Human Induced Pluripotent Stem Cells’. Nature 471 (7336): 68-73. https://doi.org/10.1038/nature09798.
Lotz, Steven, Susan Goderie, Nicolas Tokas, Sarah E. Hirsch, Faizzan Ahmad, Barbara Corneo, Sheila Le, et al. 2013. ‘Sustained Levels of FGF2 Maintain Undifferentiated Stem Cell Cultures with Biweekly Feeding’. PloS One 8 (2): e56289. https://doi.org/10.1371/journal.pone.0056289.
Madsen, Ralitsa R., James Longden, Rachel G. Knox, Xavier Robin, Franziska Völlmy, Kenneth G. Macleod, Larissa S. Moniz, et al. 2021. ‘NODAL/TGFβ Signalling Mediates the Self-Sustained Stemness Induced by PIK3CAH1047R Homozygosity in Pluripotent Stem Cells’. Disease Models & Mechanisms 14 (3): dmm048298. https://doi.org/10.1242/dmm.048298.
Martyn, Iain, Ali H. Brivanlou, and Eric D. Siggia. 2019. ‘A Wave of WNT Signaling Balanced by Secreted Inhibitors Controls Primitive Streak Formation in Micropattern Colonies of Human Embryonic Stem Cells’. Development (Cambridge, England) 146 (6): dev172791. https://doi.org/10.1242/dev.172791.
Montavon, Thomas, Nicholas Shukeir, Galina Erikson, Bettina Engist, Megumi Onishi-Seebacher, Devon Ryan, Yaarub Musa, et al. 2021. ‘Complete Loss of H3K9 Methylation Dissolves Mouse Heterochromatin Organization’. Nature Communications 12 (1): 4359. https://doi.org/10.1038/s41467-021-24532-8.
Nicetto, Dario, Greg Donahue, Tanya Jain, Tao Peng, Simone Sidoli, Lihong Sheng, Thomas Montavon, et al. 2019. ‘H3K9me3-Heterochromatin Loss at Protein-Coding Genes Enables Developmental Lineage Specification’. Science (New York, N.Y.) 363 (6424): 294-97. https://doi.org/10.1126/science.aau0583.
Onder, Tamer T., Nergis Kara, Anne Cherry, Amit U. Sinha, Nan Zhu, Kathrin M. Bernt, Patrick Cahan, et al. 2012. ‘Chromatin-Modifying Enzymes as Modulators of Reprogramming’. Nature 483 (7391): 598-602. https://doi.org/10.1038/nature10953.
Padeken, Jan, Stephen P. Methot, and Susan M. Gasser. 2022. ‘Establishment of H3K9-Methylated Heterochromatin and Its Functions in Tissue Differentiation and Maintenance’. Nature Reviews. Molecular Cell Biology 23 (9): 623-40. https://doi.org/10.1038/s41580-022-00483-w.
Puigdevall, Pau, Julie Jerber, Petr Danecek, Sergi Castellano, and Helena Kilpinen. 2023. ‘Somatic Mutations Alter the Differentiation Outcomes of IPSC-Derived Neurons’. Cell Genomics 3 (4): 100280. https://doi.org/10.1016/j.xgen.2023.100280.
Ren, Zhili, Hui Zhong, Chengcheng Song, Chunhao Deng, Hsun-Ting Hsieh, Weiwei Liu, and Guokai Chen. 2020. ‘Insulin Promotes Mitochondrial Respiration and Survival through PI3K/AKT/GSK3 Pathway in Human Embryonic Stem Cells’. Stem Cell Reports 15 (6): 1362-76. https://doi.org/10.1016/j.stemcr.2020.10.008.
Shi, Yan, Jeong Tae Do, Caroline Desponts, Heung Sik Hahm, Hans R. Schöler, and Sheng Ding. 2008. ‘A Combined Chemical and Genetic Approach for the Generation of Induced Pluripotent Stem Cells’. Cell Stem Cell 2 (6): 525-28. https://doi.org/10.1016/j.stem.2008.05.011.
Song, Chengcheng, Faxiang Xu, Zhili Ren, Yumeng Zhang, Ya Meng, Yiqi Yang, Shreyas Lingadahalli, et al. 2019. ‘Elevated Exogenous Pyruvate Potentiates Mesodermal Differentiation through Metabolic Modulation and AMPK/MTOR Pathway in Human Embryonic Stem Cells’. Stem Cell Reports 13 (2): 338-51. https://doi.org/10.1016/j.stemcr.2019.06.003.
Song, Yang, Jennifer Soto, Binru Chen, Tyler Hoffman, Weikang Zhao, Ninghao Zhu, Qin Peng, et al. 2022. ‘Transient Nuclear Deformation Primes Epigenetic State and Promotes Cell Reprogramming’. Nature Materials 21 (10): 1191-99. https://doi.org/10.1038/s41563-022-01312-3.
Sun, Li, Xiuling Fu, Gang Ma, and Andrew P. Hutchins. 2021. ‘Chromatin and Epigenetic Rearrangements in Embryonic Stem Cell Fate Transitions’. Frontiers in Cell and Developmental Biology 9:637309. https://doi.org/10.3389/fcell.2021.637309.
TeSlaa, Tara, Andrea C. Chaikovsky, Inna Lipchina, Sandra L. Escobar, Konrad Hochedlinger, Jing Huang, Thomas G. Graeber, Daniel Braas, and Michael A. Teitell. 2016. ‘α-Ketoglutarate Accelerates the Initial Differentiation of Primed Human Pluripotent Stem Cells’. Cell Metabolism 24 (3): 485-93. https://doi.org/10.1016/j.cmet.2016.07.002.
Vallier, Ludovic, Morgan Alexander, and Roger A. Pedersen. 2005. ‘Activin/Nodal and FGF Pathways Cooperate to Maintain Pluripotency of Human Embryonic Stem Cells’. Journal of Cell Science 118 (Pt 19): 4495-4509. https://doi.org/10.1242/jcs.02553.
Villa-Diaz, Luis G., Jin Koo Kim, Alex Laperle, Sean P. Palecek, and Paul H. Krebsbach. 2016. ‘Inhibition of Focal Adhesion Kinase Signaling by Integrin A6β1 Supports Human Pluripotent Stem Cell Self-Renewal’. Stem Cells 34 (7): 1753-64. https://doi.org/10.1002/stem.2349.
Vitillo, Loriana, Melissa Baxter, Banu Iskender, Paul Whiting, and Susan J. Kimber. 2016. ‘Integrin-Associated Focal Adhesion Kinase Protects Human Embryonic Stem Cells from Apoptosis, Detachment, and Differentiation’. Stem Cell Reports 7 (2): 167-76. https://doi.org/10.1016/j.stemcr.2016.07.006.
Wang, Chenfei, Xiaoyu Liu, Yawei Gao, Lei Yang, Chong Li, Wenqiang Liu, Chuan Chen, et al. 2018. ‘Reprogramming of H3K9me3-Dependent Heterochromatin during Mammalian Embryo Development’. Nature Cell Biology 20 (5): 620-31. https://doi.org/10.1038/s41556-018-0093-4.
Wang, Linlin, Thomas C. Schulz, Eric S. Sherrer, Derek S. Dauphin, Soojung Shin, Angelique M. Nelson, Carol B. Ware, et al. 2007. ‘Self-Renewal of Human Embryonic Stem Cells Requires Insulin-like Growth Factor-1 Receptor and ERBB2 Receptor Signaling’. Blood 110 (12): 4111-19. https://doi.org/10.1182/blood-2007-03-082586.
Wang, Tao, Keshi Chen, Xiaoming Zeng, Jianguo Yang, Yun Wu, Xi Shi, Baoming Qin, et al. 2011. ‘The Histone Demethylases Jhdmla/1b Enhance Somatic Cell Reprogramming in a Vitamin-C-Dependent Manner’. Cell Stem Cell 9 (6): 575-87. https://doi.org/10.1016/j.stem.2011.10.005.
Warmflash, Aryeh, Benoit Sorre, Fred Etoc, Eric D. Siggia, and Ali H. Brivanlou. 2014. ‘A Method to Recapitulate Early Embryonic Spatial Patterning in Human Embryonic Stem Cells’. Nature Methods 11 (8): 847-54. https://doi.org/10.1038/nmeth.3016.
Watanabe, Momoko, Jessie E. Buth, Jillian R. Haney, Neda Vishlaghi, Felix Turcios, Lubayna S. Elahi, Wen Gu, et al. 2022. ‘TGFβ Superfamily Signaling Regulates the State of Human Stem Cell Pluripotency and Capacity to Create Well-Structured Telencephalic Organoids’. Stem Cell Reports 17 (10): 2220-38. https://doi.org/10.1016/j.stemcr.2022.08.013.
Xu, Ruimin, Sen Li, Qiu Wu, Chong Li, Manxi Jiang, Lei Guo, Mo Chen, et al. 2022. ‘Stage-Specific H3K9me3 Occupancy Ensures Retrotransposon Silencing in Human Pre-Implantation Embryos’. Cell Stem Cell 29 (7): 1051-1066.e8. https://doi.org/10.1016/j.stem.2022.06.001.
Yu, Yongxin, Peng Deng, Bo Yu, John M. Szymanski, Tara Aghaloo, Christine Hong, and Cun-Yu Wang. 2017. ‘Inhibition of EZH2 Promotes Human Embryonic Stem Cell Differentiation into Mesoderm by Reducing H3K27me3’. Stem Cell Reports 9 (3): 752-61. https://doi.org/10.1016/j.stemcr.2017.07.016.
Zhu, Jiang, Mazhar Adli, James Y. Zou, Griet Verstappen, Michael Coyne, Xiaolan Zhang, Timothy Durham, et al. 2013. ‘Genome-Wide Chromatin State Transitions Associated with Developmental and Environmental Cues’. Cell 152 (3): 642-54. https://doi.org/10.1016/j.cell.2012.12.033.

1. A method of standardising pluripotent stem cells (PSCs), the method comprising culturing at least one PSC in cell culture medium supplemented with an inhibitor of a methyltransferase that catalyses the mono-, di- or tri-methylated states of histone H3 at lysine residue 9 (H3K9 MTase), a H3K9 MTase inhibitor.

2. The method as claimed in claim 1, wherein the at least one PSC is at least one of an induced pluripotent stem cell (iPSC), a naturally occurring pluripotent stem cell (PSC) or embryonic stem cell (ESC).

3. The method as claimed in claim 2, wherein the at least one PSC is an iPSC.

4. The method according to any one of the preceding claims, wherein the H3K9 MTase is at least one of G9a, SUV39H1, SUV39H2, SETDB1, SETDB2, GLP, PRDM2 or ASH1L.

5. The method as claimed in claim 4, wherein the H3K9 MTase is G9a.

6. The method according to any one of the preceding claims, wherein the H3K9 MTase inhibitor is at least one of BIX01294, BIX01338, UNC0224, UNCO0638, UNCO0321 or A-366.

7. The method as claimed in claim 6, wherein the H3K9 HMTase inhibitor is BIX01294.

8. The method according to any one of the preceding claims, wherein the at least one PSC is an iPSC, the H3K9 MTase is G9a and the H3K9 HMTase inhibitor is BIX01294.

9. The method according to any one of the preceding claims, wherein the at least one PSC is cultured in an at least a one, two, three, four or five step cell culture process.

10. The method as claimed in claim 9, wherein the at least one PSC is cultured in a three step cell culture process.

11. The method as claimed in claim 9 or claim 10, wherein when there is more than one step, a subsequent step comprises culturing the at least one PSC from a previous step in a new cell culture medium.

12. The method as claimed in any one of claims 9 to 11, wherein when there is more than one step, only one step comprises cell culture medium supplemented with the H3K9 MTase inhibitor.

13. The method as claimed in claim 12, wherein the first step comprises cell culture medium supplemented with the H3K9 MTase inhibitor.

14. The method according to any one of the preceding claims, wherein the cell culture medium, or at least one of the cell culture media, is/are further supplemented with an inhibitor of histone demethylation.

15. The method according to any one of the preceding claims, wherein the inhibitor of histone demethylation is at least one of tranylcypromine, RN-1, GSK2879, S2101, OG-L002, T-3775440 HCl, Iadademstat (ORY-1001) 2HCl, SP2509, GSK2879552 2HCl, Pulrodemstat (CC-90011) besylate, GSK-LSD1 2HCl or LSD1-C76.

16. The method as claimed in claim 15, wherein the inhibitor of histone demethylation is tranylcypromine.

17. The method according to any one of the preceding claims, wherein the cell culture medium, or at least one of the cell culture media, is/are further supplemented with, or supplemented with, one or more small molecules with the following biological activity:
(i) glycogen kinase inhibitor;
(ii) selective inhibitor of Rho-associated, coiled-coil containing protein kinase (ROCK);
(iii) MAPK inhibitor;
(iv) Wnt inhibitor;
(v) B-raf inhibitor; and
(vi) histone deacetylase (HDAC) inhibitor.

18. The method as claimed in claim 17, wherein:
(i) glycogen kinase inhibitor is selected from the group consisting of CHIR99021, SB-216763; CHIR 99021 trihydrochloride, BIO-acetoxime, GSK-3β Inhibitor XII, GSK-3 Inhibitor XV, TD114-2, TD114-3, IM12, CHIR98014, AT7519, TWS119, Tideglusib (NP031112), AZD1080, AR-A014418, TDZD-8, LY2090314, WAY-119064, KY19382 (A3051), BRD0705, 1-Alsterpaullone, IM-12 and SB-415286;
(ii) the Rho-associated, coiled-coil containing protein kinase (ROCK) inhibitor is selected from the group consisting of Y-27632, Fasudil, GSK429286A, H-1152 dihydrochloride, Azaindole 1 (TC-S 7001), RKI-1447, Y-39983 HCl, Ripasudil (K-115) hydrochloride dihydrate, Hydroxyfasudil (HA-1100) HCl, Netarsudil (AR-13324) 2HCl, ZINC00881524, thiazovivin, Y-33075, PT-262, CAY10746, Cotosudil, WF-536 and AS1892802;
(iii) the MAPK inhibitor is selected from the group consisting of PD0325901, AZD8330; Trametinib (GSK1120212), U0126, PD184352 (CI-1040), Refametinib (RDEA119), BI-847325,TAK-733, Binimetinib (MEK162), GDC-0623, E6201 (ER-806201), PD 198306, RO4987655, AZD8330 (ARRY-424704), Refametinib (BAY 869766; RDEA119), MAP855, SL327, Selumetinib (AZD6244), Cobimetinib, Trametinib (GSK1120212; JTP-74057), Pimasertib (AS703026), PD318088, Avutometinib, U0126, PD-334581, PD98059, U0124, PD184161, EBI-1051, Zapnometinib (PD0184264), Temuterkib (LY3214996) and Rineterkib;
(iv) the Wnt inhibitor is selected from the group consisting of IWP-2, WNT-C59, XAV-939, CCT251545, IWP 12, IWP L6, Wnt-C59, IWR-1, LGK974 (WNT974), IWP-3, TC-E 5001, IWP-4, IWP-01, JW67, OM-153, YW1128, KY02111, GNF-6231 and DK419;
(v) the B-raf inhibitor is selected from the group consisting of SB590885, Sorafenib, Vemurafenib (PLX4032), PLX-4720, Dabrafenib (GSK2118436), Regorafenib (BAY 73-4506), RAF265 (CHIR-265), TAK-632, Agerafenib (RXDX-105), GNE-9815, L-779450, B-Raf inhibitor 1 (Compound 13) dihydrochloride, Belvarafenib (HM95573), B-Raf IN 1, AZ304, PLX8394, RAF709, Lifirafenib (BGB-283), CCT196969, LY3009120, Avutometinib, GDC0879, Tinlorafenib (PF-07284890), SHR902275, Everafenib, AZ304, GDC-0879, HG6-64-1, LUT014 and CFT1946; and
(vi) the HDAC class 1 inhibitor (HDAC1, 2, 3, 8) is selected from the group consisting of valproic acid (VPA), Mocetinostat (MGCD0103, MG0103), (−)-Parthenolide, Suberohydroxamic acid, Domatinostat (4SC-202), UF010, Abexinostat (PCI-24781, CRA-024781), Romidepsin (FK228, Depsipeptide, FR 901228, NSC 630176), SR-4370, TC-H 106 (Pimelic Diphenylamide 106), Tacedinaline (CI994, PD-123654, GOE-5549, Acetyldinaline), BRD3308, Entinostat (MS-275, SNDX-275), Suberohydroxamic acid (suberic bishydroxamic acid) and BG45.

19. The method according to any one of the preceding claims, wherein the cell culture medium, or at least one of the cell culture media, is/are further supplemented with, or supplemented with at least one of CHIR99021, Y-27632, PD0325901, IWP-2, SB590885 and VPA.

20. The method according to any one of the preceding claims, wherein the cell culture medium, or at least one of the cell culture media, is/are further supplemented with one or more of the following:
(i) N2 supplement;
(ii) B27 supplement;
(iii) GlutaMax;
(iv) MEM Nonessential amino acids;
(v) penicillin-streptomycin;
(vi) L-Ascorbic acid 2-phosphate; and
(vii) heregulinβ-1 (HRG).

21. The method according to any one of the preceding claims, wherein the cell culture medium, or at least one of the cell culture media, is/are further supplement with at least one of an inhibitor of histone methyltransferase EZH2 and/or a Dot1L inhibitor.

22. The method as claimed in claim 21, wherein the inhibitor of histone methyltransferase EZH2 is DZNep.

23. The method as claimed in claim 21 or claim 22, wherein the Dot1L inhibitor is EPZ004777.

24. The method as claimed in any one of claims 9 to 23, wherein when there is more than one step, only one step comprises cell culture medium supplemented with the H3K9 MTase inhibitor, the inhibitor of histone demethylation and the inhibitor of histone methyltransferase EZH2.

25. The method as claimed in claim 24, wherein the first step comprises cell culture medium supplemented with the H3K9 MTase inhibitor, the H3K9 MTase inhibitor, the inhibitor of histone demethylation and the inhibitor of histone methyltransferase EZH2.

26. The method according to any one of the preceding claims, wherein the media, or when the at least one SC is cultured in at least two, three, four or five step cell culture process, the cell culture medium used in the first step, is further supplement with at least one of an inhibitor of histone methyltransferase EZH2 and/or a Dot1L inhibitor.

27. The method according to any one of the preceding claims, wherein when the at least one PSC is cultured in at least two, three, four or five step cell culture process, the cell culture medium used in the last step, is further supplemented with basic Fibroblast Growth Factor (bFGF) and/or AlbuMax-II supplement.

28. The method as claimed in any one of claims 9 to 27, wherein when the at least one SC is cultured in a three step procedure:
(i) the cell culture medium used in the first step comprises N2 supplement, B27 supplement, GlutaMax, NEAA, penicillin-streptomycin, L-ascorbic acid-2-phosphate, HRG, CHIR99021, Y-27632, PD0325901, IWP-2, SB590885, VPA, tranylcypromine, DZNep, EPZ004777 and BIX01294;
(ii) the cell culture medium used in the second step comprises N2 supplement, B27 supplement, GlutaMax, NEAA, penicillin-streptomycin, L-ascorbic acid-2-phosphate, HRG, CHIR99021, Y-27632, PD0325901, IWP-2, SB590885; and
(iii) the cell culture medium used in the third step comprises N2 supplement, B27 supplement, GlutaMax, NEAA, penicillin-streptomycin, L-ascorbic acid-2-phosphate, HRG, CHIR99021, Y-27632, PD0325901, IWP-2, SB590885, bFGF and AlbuMax-II supplement.

29. The method according to any one of the preceding claims, wherein the at least one SC is maintained in each step for a period of time of at least 1, 2, 3, 4, 5, 6, 7, 8, 9, 10, 11, 12, 13, 14, 15, 16, 17, 18, 19 or 20 days.

30. The method as claimed in claim 29, wherein when there is more than one step, each step is for the same, or for a different, period of time.

31. A cell obtainable by the method according to any one of the preceding claims.

32. A cell as claimed in claim 31, wherein the cell express SOX2, OCT4 and NANOG.

33. A cell as claimed in claim 31 or claim 32, wherein the cell is ectoderm, mesoderm or endoderm competent.

34. A cell as claimed in any one of claims 31 to 33, wherein the cell is cerebral, renal or digestive tract organoid competent.

35. Use of a cell as claimed in any one of claims 31 to 34, in the development of an organoid, preferably wherein the organoid is a cerebral, renal or digestive tract organoid.

36. Use of a cell as claimed in any one of claims 31 to 34, as a tool for research.

37. Use of a cell as claimed in any one of claims 31 to 34, as a model for disease.

38. Use of a cell as claimed in any one of claims 31 to 34, in the discovery of a therapeutic.

39. Use of a cell as claimed in any one of claims 31 to 34, in the production of an organoid, preferably wherein the organoid is a cerebral, renal or digestive tract organoid.

40. An organoid obtainable by the use as claimed in claim 39.

41. A cell as claimed in any one of claims 31 to 34, or an organoid as claimed in claim 40, for use in therapy, surgery or diagnosis.

42. A method of treating, ameliorating or preventing a disease or medical condition in a subject, the method comprising administering, to a subject in need thereof, a therapeutically effective amount of a cell as claimed in any one of claims 31 to 34 or an organoid as claimed in claim 40.

43. A use as claimed in any one of claims 35 to 39, a cell or organoid for use as claimed in claim 41, or a method as claimed in claim 42, wherein the cell has undergone differentiation prior to use or application in the method.

44. A cell culture medium or media as defined in any one of claims 1 to 30.

45. A kit comprising a cell culture medium or media as claimed in claim 44.
